(19) United States
(12) Patent Application Publication
(10) Pub. No.: US 2026/0335582 A1
(43) Pub. Date: Oct. 29, 2026

(54) Spring Lamina Valve and Nasal Cannula Incorporating Such a Valve

(71) Applicant: OXFO Corporation, Chestnut Hill, MA (US)

(72) Inventors: Carlos Fernando Bazoberry, Chestnut Hill, MA (US); Brent H. Young, Boston, MA (US)

(21) Appl. No.: 19/560,870

(22) Filed: Mar. 9, 2026

Related U.S. Application Data
(63) Continuation-in-part of application No. 18/371,097, filed on Sep. 21, 2023, now abandoned.
(60) Provisional application No. 63/408,653, filed on Sep. 21, 2022.

(51) Int. Cl. A61M 16/06; A61M 16/20

(52) U.S. Cl. CPC A61M 16/0672; A61M 16/208; A61M 2202/0007; A61M 2202/0208; A61M 2205/0216; A61M 2210/0618; A61M 2210/0625

(57) ABSTRACT

A nasal cannula with a spring lamina valve for resiliently deflecting toward the nares of a patient during inhalation to prevent unintended entrainment of ambient air while permitting exhalation through and around the nasal prongs of the nasal cannula by a resilient deflection of the spring lamina valve away from the nares of the patient. Left and right outer flaps of resiliently flexible, biocompatible material have nasal prong apertures, and left and right inner flaps are resiliently and hingedly coupled thereto by isthmuses that tend to bow the left and right outer flaps into a saddle contour matching the contour of the nose of the patient when the left and right nasal prongs are respectively received through the left and right apertures. The left and right outer flaps can be unitarily formed with a connecting bridge portion, or they can be separate members.

### RELATED APPLICATIONS

This application is a continuation-in-part of Application No. 18/371,097, filed Sep. 21, 2023, which in turn claims priority to Provisional Application No. 63/408,653, filed 09/21/2022, both incorporated herein by reference.

### FIELD OF THE INVENTION

The present invention relates generally to the delivery of gases from a source to a recipient. More particularly, disclosed herein is lamina valve and a nasal cannula incorporating such a valve for use within a system for delivering oxygen at ambient pressure from a donor reservoir to a recipient wherein a nasal cannula with such a lamina valve is operative to permit inhalation of oxygen from the donor reservoir with a controlled fraction of inspired oxygen (FiO2) while permitting ready exhalation.

### BACKGROUND OF THE INVENTION

Normally, the lungs absorb oxygen in sufficient supply from the air during natural breathing. However, certain conditions can prevent a person from getting enough oxygen. As a result, oxygen therapy with oxygen delivery equipment is required. Patients can receive oxygen therapy from a source of oxygen through tubes resting in their nose, through a facemask, or through a tube placed in their trachea or windpipe. Oxygen treatment increases the amount of oxygen the lungs receive and deliver to the blood. Oxygen therapy may be prescribed for a patient when the patient has a condition that causes the patient’s blood oxygen levels to be too low. Low blood oxygen may make patients feel short of breath, tired, or confused and can damage the patient’s body. Oxygen therapy may be needed on a temporary basis, such as due to a treatable respiratory illness, or on a long-term basis. Often, the source of oxygen is a tank of compressed oxygen gas or liquid.

The supply of oxygen can be a critical need for hospital patients and others. Meanwhile, in developing countries and during times of increased demand in all places, shortages of oxygen and excessive costs can place extreme limits on availability and can jeopardize the health and safety of patients in need. For instance, during the COVID-19 pandemic, the demand for oxygen left hospitals and other caregiving institutions in dire need of the life-saving gas. One headline from the AP News network on June 24, 2020 warned, “Scarce Medical Oxygen Worldwide Leaves Many Gasping for Life.” One day later, Reuters observed, “WHO Warns of Oxygen Shortage as COVID Cases Set to Top 10 Mln” with the World Health Organization estimating based on there being approximately one million new coronavirus cases worldwide per week that the world will need 620,000 cubic meters of oxygen per day, which roughly equaled 88,000 large cylinders, for COVID-19 patients alone.

One way that supplemental oxygen is supplied to patients under the teachings of the prior art is via a fluidic connection, typically tubing, between a pressurized source of oxygen, such as an oxygen cylinder or tank, and an output interface, such as a nasal cannula or mask, to the patient to provide high flows of oxygen. High-flow systems inherently and necessarily provide an excess supply of oxygen to ensure that the patient has sufficient oxygen over the entire respiratory cycle. In such systems, the oxygen flows continuously, regardless of whether the patient is breathing in or out. As a result, even while the patient exhales and cannot intake oxygen, the oxygen flows constantly. Huge volumes of oxygen are thus wasted. Indeed, half or even more of the constantly supplied oxygen is wasted and is simply expelled to the atmosphere. Indeed, a portion of the supplied oxygen is often wasted during inhalation, and the entirety of the continuously supplied oxygen is wasted during exhalation.

Concomitantly, it will be recognized that the ability to conserve oxygen with one patient may well save the life of another, particularly in exigent circumstances, such as during an epidemic or a pandemic involving respiratory distress where need can dangerously outpace supply. In remote and economically challenged locations, replenishing oxygen supplies can be highly costly or even catastrophically impossible. The challenge of providing oxygen in sufficient supply while minimizing waste is well recognized.

In a typical nasal cannula configuration, one end of an oxygen supply tube is connected to the source of oxygen while the other end of the tubing splits into two branches that meet to form a loop. Two nasal prongs are positioned along the loop for insertion into a patient's nares. Oxygen continuously flows through the tubing to exit through the nasal prongs and into the patient's nares. During inspiration, the patient thus inhales oxygen through the prongs, often together with entrained room air that is drawn through the space between the nasal prongs and the walls of the patient's nares. During exhalation, the patient exhales through the space between the nasal prongs and the walls of the patient's nares while oxygen continues to exit into the patient's nares. Much of that oxygen is carried with the expiratory flow into the surrounding room air. Indeed, high-flow nasal cannula systems rely on the gaps around the nasal prongs of the cannula to vent excess flow during both inspiration and expiration and to permit patient expiration.

In continuous flow systems, the fraction of inspired oxygen (Fi02) provided to the patient is sought to be controlled by increasing or decreasing the oxygen flow through the oxygen supply tube. Disadvantageously, once the flow rate is set, it works optimally only for the breathing pattern of the patient at the time of calibration. Changes to that breathing pattern, such as through physical exertion or another change in circumstance, will affect the FiO2, which will in turn affect the saturation of the patient. For example, when a patient takes a deeper breathe or is exerting and breathing more frequently as a result of exertion, the patient will inhale a larger volume into his or her lungs. Since the volume of oxygen provided is fixed but the volume of air is not, more air enters the blend, more dilution occurs, and FiO2 is reduced. The patient’s oxygen-blood saturation (SaO2) may thus decrease with decreased FiO2. Active readjustment from the patient or medical personnel may be required. However, patients may not possess the skills or knowledge necessary to make such adjustments accurately, or they may simply be unaware or inattentive to the need to do so. Moreover, medical personnel may be unavailable to make continual adjustments to oxygen flow, particularly in non-medical settings.

In attempting to confront the foregoing, pulsed oxygen delivery systems have been disclosed to attempt to conserve oxygen by sensing the patient's breathing cycle and delivering a short-duration flow or pulse of oxygen during inhalation. In pulsed oxygen delivery, oxygen is “pulsed” to the patient in one bolus of oxygen during the inhalation phase. However, such systems rely on complex circuitry and operation and may not adequately approximate natural human breathing. Multiple important factors come into play in supplying consistent and effective supplemental oxygen to patients. By way of example and not limitation, FiO2 is affected by oxygen purity, the trigger mechanism, the pulsed dose, the duration of the pulse, the pulsed flow curve, the ventilation rate, and inspiratory peak flow. No known device has optimized all of these factors perfectly. Instead, each manufacturer is forced to make trade-offs among them.

Pulsed oxygen delivery systems are typically set to trigger the pulse dose upon a specific but non-standardized negative pressure in the inspiratory curve of the breathing cycle. If the triggering pressure is too low, then the pulse dose will be delivered too early in the inspiratory curve when a patient does not have enough negative pressure to inhale the full amount of oxygen delivered. If the triggering pressure is too high, then the pulse is delivered too late in the inspiratory curve for optimal clinical use. Highly complex engineering, hardware, and software would be required for pulse oxygen delivery systems to respond based on real-time biofeedback from the patent to respond dynamically and with greater sensitivity to negative pressure and the patient’s respiratory cycle and volume. To be fully responsive, the system would need to respond immediately or nearly immediately in what can be referred to as a “shadow effect” to meet the fluctuating needs of daily life, such as when a person transitions from sitting, to standing, to walking, to talking, to exercising, and other daily tasks. Current systems do not do this satisfactorily. Further, it will again be noted that current supplemental oxygen systems typically require the patient to remember to adjust the rate of flow and to do so based on what essentially amounts to a best guess based on changing activity levels or respiratory rates. These and further factors render patients using pulsed oxygen systems vulnerable to desaturation and resulting discomfort and health risks.

With a knowledge of the concomitant yet competing needs for conserving oxygen while being able to provide ample volumes of the same on demand, the present inventors developed the Automatic System for the Conservation of Gas and other Substances of Patent No. 12,214,126, which is incorporated herein by reference. That automatic conservation system is operative to provide ample oxygen to patients on demand while conserving against loss and waste, including during the expiratory breathing phase. The automatic conservation system minimizes the oxygen consumption of individual patients while meeting patient needs and maximizing the effective supply of oxygen. In so doing, the automatic conservation system enables better health outcomes in a cost-efficient manner, even in times of public health crises.

In practices of the system of the ‘126 patent, an expandable and compressible donor reservoir retains a volume of oxygen at ambient pressure. A supply conduit receives oxygen from a source of oxygen to fill the donor reservoir, and an ambient pressure conduit supplies oxygen from the donor reservoir to a patient. An inflation detection system detects when the donor reservoir is inflated with oxygen to a predetermined state of inflation, such as within a range of a fully inflated condition, and when the donor reservoir is below the predetermined state of inflation. When the donor reservoir is inflated to the predetermined state of inflation, the valve system is closed to prevent oxygen from flowing from the source of oxygen and into the donor reservoir. When the donor reservoir is below the predetermined state of inflation, the valve system is opened to permit oxygen to flow from the source of oxygen to replenish the donor reservoir automatically. Oxygen can thus be continuously retained in the reservoir and supplied on demand to a patient through a patient interface delivery device, such as a nasal cannula or a breathing mask, with minimized waste. By providing such an on-demand oxygen supply system in place of, for instance, a high-flow oxygenation system, massive reductions in oxygen requirements and waste are realized.

However, the present inventors have further realized that supplying oxygen at ambient pressure in an on-demand format establishes unique needs and opportunities for functionality at the actual patient interface, such as the nasal cannula. For instance, while high-flow nasal cannula systems rely on the gaps around the nasal prongs of the cannula to vent excess flow even during inspiration, such an architecture is generally incompatible with permitting a patient to inspire naturally from an ambient pressure reservoir since those gaps provide a low-resistance path for the entrainment of ambient air thereby leading to unintended FiO2 dilution of the supplied oxygen. It is thus desirable in an ambient pressure system as taught by the inventors’ own Patent No. 12,214,126 where oxygen is drawn from an ambient pressure reservoir through a nasal cannula to prevent the unintended entrainment of ambient air through gaps around the nasal prongs of the cannula. The inventors also recognize that, where oxygen is naturally inspired from a donor reservoir at ambient pressure through an ambient pressure conduit, it is desirable for the recipient to be able to exhale freely, including through the cannula itself. It is further desirable to prevent the breath so exhausted from being returned into the ambient pressure conduit and potentially into the donor reservoir to mix with the oxygen retained therein. The inventors have further appreciated that it would be advantageous in such an on-demand system to be able to exercise direct and immediate control over the ratio of oxygen and entrained air inhaled through the nasal cannula during inspiration.

### SUMMARY OF THE INVENTION

In view of the foregoing, the present inventors further set forth with the basic object of providing a nasal cannula particularly adapted for use with an ambient-pressure oxygen dispensing and conservation system.

A further object of embodiments of the invention is to provide a nasal cannula for an ambient-pressure oxygen dispensing and conservation system that facilitates the provision of an ample supply of oxygen on-demand while minimizing or eliminating inefficient oxygen losses.

A more specific object of embodiments of the invention is to provide a lamina valve for a nasal cannula and a nasal cannula with such a lamina valve that prevents the unintended entrainment of ambient air through gaps around the nasal prongs of the cannula in an ambient pressure oxygen dispensing and conservation system.

A related object of practices of the invention is to provide a lamina valve for a nasal cannula and a nasal cannula with such a lamina valve that reduces volatility and unpredictability in FiO2 due to the uncontrolled entrainment of ambient air during inspiration.

Another particular object of embodiments of the invention is to provide a nasal cannula in an ambient pressure oxygen dispensing and conservation system that permits breath to be exhausted directly through the cannula.

Another particular object of embodiments of the invention is to provide a nasal cannula in an ambient pressure oxygen dispensing and conservation system that is operative to prevent breath from being exhaled into a connected ambient pressure conduit.

Yet another object of the invention in particular embodiments is to provide a nasal cannula that permits direct and immediate control over the ratio of oxygen and entrained air inhaled through the nasal cannula.

An underlying object of the invention with respect to the ratio of oxygen and entrained air inhaled through the nasal cannula is to prevent the unintended entrainment of ambient air through gaps around the nasal prongs of the cannula thereby facilitating reliable control over the fraction of inspired oxygen (FiO2) provided to the patient.

A further object of the invention is to provide a low-resistance, comfort-preserving seal that suppresses room-air entrainment during inhalation in an elegant manner without requiring an intranasal plug-seal and without relying on positive pressure at the patient interface.

These and further objects and advantages of the present invention will become obvious not only to one who reviews the present specification and drawings but also to those who have an opportunity to experience the lamina valve for a nasal cannula, a nasal cannula incorporating such a valve, and an ambient pressure oxygen dispensing and conservation system using such a cannula in operation. However, it will be appreciated that, while the accomplishment of plural of the foregoing objects in a single embodiment of the invention may be possible and indeed preferred, not all embodiments will seek or need to accomplish each and every potential advantage and function. Nonetheless, all such embodiments should be considered within the scope of the present invention.

One will appreciate that the foregoing discussion broadly outlines the more important goals and features of the invention to enable a better understanding of the detailed description that follows and to instill a better appreciation of the inventors’ contribution to the art. Before any particular embodiment or aspect thereof is explained in detail, it must be made clear that the following details of construction and illustrations of inventive concepts are mere examples of the many possible manifestations of the invention.

In carrying forth one or more of the foregoing objects, a nasal cannula is taught herein for use with an ambient pressure gas dispensing system with ambient pressure tubing for cooperating in providing gas to an individual. In one embodiment, the nasal cannula is founded on a nasal cannula body with an inner volume. First and second nasal prongs extend from the nasal cannula body. The first and second nasal prongs are in fluidic communication with the inner volume of the nasal cannula body. A gas reception aperture is in fluidic communication with the inner volume of the nasal cannula body for receiving gas from the tubing of the gas dispensing system, and a one-way expiratory valve is retained by the nasal cannula body in fluidic communication with the inner volume of the nasal cannula body. Under this construction, gas provided by the tubing can be inhaled through the nasal prongs and, during expiration, expired breath can be discharged through the one-way expiratory valve.

In certain embodiments, the nasal cannula can further comprise an FiO2 adjustment aperture in the nasal cannula body that is in fluidic communication with the inner volume of the nasal cannula body. The FiO2 adjustment aperture is selectively adjustable in size, such as by actuation of a movable cover. The size of the FiO2 adjustment aperture can thus be adjusted to provide a desired entrainment of atmospheric air with gas inhaled through the first and second nasal prongs. Still more particularly, embodiments of the nasal cannula are disclosed wherein there are first and second FiO2 adjustment apertures in the nasal cannula body with the first and second FiO2 adjustment apertures being individually and selectively adjustable in size.

According to practices of the invention, the nasal cannula body can be formed from a first body member assembled with a second body member. The first body member can have a central portion that defines a reception cavity, and the second body member can be at least partially received into the reception cavity of the first body member, such as by being received therethrough. For instance, the reception cavity of the first body member can be generally tubular, and the second body member can have a corresponding generally tubular portion. To facilitate the retention of the nasal cannula relative to the head of a wearer, first and second straps can extend laterally in opposite directions from the central portion of the first body member.

As disclosed herein, the first body member can be formed from a resilient and substantially flexible material, and the second body member can be formed from a substantially rigid material. In embodiments of the invention, the second body member has an inner volume with an opening bounded by a platform, and the first body member has a nasal prong platform. The nasal prongs extend from the nasal prong platform of the first body member, and the nasal prong platform of the first body member establishes a sealing engagement with the platform of the second body member when the first and second body members are assembled. The sealing engagement is, in part, facilitated by the resilient and flexible nature of the first body member.

Where the nasal cannula is formed with first and second body members, the gas reception aperture can be disposed at a first end of the second body member, and the one-way expiratory valve can be characterized as a first one-way expiratory valve disposed at a second end of the second body member. In such embodiments, a second one-way expiratory valve and potentially a third one-way expiratory valve can additionally be disposed centrally on the second body member in general alignment with at least one of the first and second nasal prongs.

Embodiments of the invention can alternatively be described as an ambient pressure gas dispensing system for providing gas at ambient pressure to an individual. The ambient pressure gas dispensing system includes a donor reservoir adapted to retain gas substantially at ambient pressure that provides gas to an individual through a nasal cannula and ambient pressure tubing interposed between the nasal cannula and the donor reservoir. A supply valve is disposed in fluidic association with the donor reservoir. The supply valve has an open condition wherein gas is allowed to flow into the donor reservoir and a closed condition wherein gas is not allowed to flow into the donor reservoir. An inflation detection system is operative to detect when the donor reservoir is inflated to within a predetermined range of a fully inflated condition. The inflation detection system detects a first condition when the donor reservoir is inflated to within the predetermined range of the fully inflated condition and a second condition when the donor reservoir is inflated below the predetermined range of the fully inflated condition. The inflation detection system is operative to actuate the supply valve to the open condition when the donor reservoir is inflated below the predetermined range of the fully inflated condition to allow replenishing gas to flow from a source of gas, such as a pressurized tank. The ambient pressure tubing has a first end and a second end. The first end of the ambient pressure tubing is in fluidic communication with the donor reservoir, and the second end of the ambient pressure tubing is fluidically connected to the nasal cannula.

In the ambient pressure gas dispensing system, the nasal cannula can again be founded on a nasal cannula body with an inner volume. First and second nasal prongs extend from the nasal cannula body in fluidic communication with the inner volume of the nasal cannula body. A gas reception aperture is provided for receiving gas from the donor reservoir through the ambient pressure tubing, and a one-way expiratory valve is retained by the nasal cannula body. The one-way expiratory valve is in fluidic communication with the inner volume of the nasal cannula body whereby gas provided by the tubing can be inhaled through the nasal prongs and whereby, during expiration, expired breath can be discharged through the one-way expiratory valve. An FiO2 adjustment aperture can be disposed in the nasal cannula body in fluidic communication with the inner volume of the nasal cannula body. The FiO2 adjustment aperture is selectively adjustable in size. With this, the size of the FiO2 adjustment aperture can be adjusted to provide a desired entrainment of atmospheric air with gas inhaled through the first and second nasal prongs.

In practices of the invention disclosed herein, the system for the conservation of oxygen supplied to a patient has an expandable and compressible donor reservoir that has an outer wall, an inner volume for retaining a volume of oxygen, and at least one orifice for allowing a passage of oxygen into and out of the inner volume. The donor reservoir can, for example, comprise a shell of flexible material, such as a shell of foil. A supply conduit is adapted to receive oxygen from a source of oxygen. The supply conduit has a first end for supplying oxygen to the donor reservoir and a second end for being fluidically connected to the source of oxygen, and an ambient pressure conduit is adapted to supply oxygen along a fluid path from the donor reservoir to a recipient, such as through a nasal cannula or a breathing mask. The ambient pressure conduit has a first end in fluidic communication with the donor reservoir, such as through a connector, for receiving oxygen from the donor reservoir and a second end for being fluidically connected to the recipient.

An inflation detection system is operable to detect a first condition in which the donor reservoir is inflated with oxygen to a predetermined state of inflation and a second condition wherein the donor reservoir is below the predetermined state of inflation. Finally, a valve system is disposed between the source of oxygen and the donor reservoir. The valve system is operative in a closed condition to prevent oxygen from flowing from the source of oxygen and into the donor reservoir when the donor reservoir is in the first condition, and the valve system is operative in an open condition to permit oxygen to flow from the source of oxygen and into the donor reservoir when the donor reservoir is in the second condition. Under this construction, oxygen can be supplied to a patient, such as through a patient breathing mask as the recipient, from the donor reservoir, and the donor reservoir can be automatically replenished to the predetermined state of inflation.

In practices of the system, the valve system and the inflation detection system are operative to maintain the volume of oxygen in the donor reservoir substantially at ambient pressure. For instance, the donor reservoir can be considered to have a fully inflated condition, and the inflation detection system can be operative to detect when the donor reservoir is inflated to within a predetermined range of the fully inflated condition. The inflation detection system can then detect the first condition when the donor reservoir is inflated to within the predetermined range of the fully inflated condition, and the inflation detection system can detect the second condition when the donor reservoir is inflated below the predetermined range of the fully inflated condition.

In certain embodiments, the inflation detection system comprises an electro-mechanical system. For instance, the inflation detection system can comprise a switch disposed to be moved by the outer wall of the donor reservoir when the donor reservoir is inflated with oxygen to the predetermined state of inflation. The switch can be biased, such as by gravity, by a resiliently compressible member, or by any other effective method, toward the donor reservoir. The switch can be considered to have an activated state wherein the switch is disposed at or beyond an inward position with respect to the inner volume of the donor reservoir and a deactivated state when the switch is moved outwardly by the outer wall of the donor reservoir when the volume of oxygen in the donor reservoir reaches the predetermined state of inflation. The valve system is operative to prevent oxygen from flowing from the source of oxygen and into the donor reservoir when the switch is in the deactivated state, and the valve system is operative to permit oxygen to flow from the source of oxygen and into the donor reservoir when the switch is in the activated state.

In particular manifestations of the system, the switch comprises a float switch. For example, the float switch can have a contact structure with a collar that is extendable and retractable relative to a central column. The collar can then retain a magnet, and the central column can then retain electrical contacts that are brought into electrical contact by a proximity of the magnet when the switch is in the activated state.

According to practices of the system, the valve system can take the form of a solenoid valve that is in electrical communication with the inflation detection system. The solenoid valve can be induced by the inflation detection system to a closed condition to prevent the flow of oxygen from the source of oxygen to the donor reservoir when the donor reservoir is in the first condition, and the solenoid valve can be induced by the inflation detection system to an open condition to permit the flow of oxygen from the source of oxygen to the donor reservoir when the donor reservoir is in the second condition.

In certain embodiments, the donor reservoir is disposed within a housing, which could comprise a main housing of the system, a sub-housing within a main housing, or some other type of housing. In other practices, the donor reservoir can be disposed without a housing. Where a housing is provided, the inflation detection system can comprise an electro-mechanical system with a switch supported by the housing and disposed to be moved by the outer wall of the donor reservoir when the donor reservoir is inflated with oxygen to the predetermined state of inflation. In alternative practices of the invention, the inflation detection system comprises a contactless detection system. For instance, the inflation detection system can take the form of an optical detection system. In certain embodiments, all or part of the housing can be transparent. With that, the state of inflation of the donor reservoir can be visually perceived, which can be of further assurance to the user that the system is in proper operation.

Embodiments of the system can further incorporate a one-way inspiratory valve disposed along the fluid path from the donor reservoir to the recipient. The one-way inspiratory valve can be operative to enable oxygen to flow from the donor reservoir, through the ambient pressure conduit, and to the recipient but to prevent reverse flow of oxygen.

A recipient delivery device, such as the nasal cannula, a patient breathing mask, or another recipient delivery device, is coupled to the second end of the ambient pressure conduit. The nasal cannula is constructed to permit breath to be exhausted directly through the cannula and to prevent exhausted breath from being returned into the connected ambient pressure conduit. As taught herein, the nasal cannula permits direct and immediate control over the ratio of oxygen and entrained air inhaled through the nasal cannula during inspiration.

Recognizing that it is highly desirable in an ambient pressure system as taught by the inventors’ Patent No. 12,214,126 to prevent the unintended entrainment of ambient air through gaps around the nasal prongs of the cannula, the present inventors have additionally developed a spring lamina valve and a nasal cannula that is deployed in combination with such a spring lamina valve that prevent the unintended entrainment of ambient air while permitting ready exhalation through and around the nasal prongs of the nasal cannula. In one such embodiment of the invention, a nasal cannula is provided with a body with an inner volume, left and right nasal prongs that extend from the nasal cannula body, the left nasal prong configured for being received into the left nare of the individual and the right nasal prong configured for being received into the right nare of the individual. The left and right nasal prongs are in fluidic communication with the inner volume of the nasal cannula body while a gas reception aperture is in fluidic communication with the inner volume of the nasal cannula body for receiving gas from the tubing of the gas dispensing system. A one-way expiratory valve is retained by the nasal cannula body in fluidic communication with the inner volume of the nasal cannula body. The spring lamina valve of the combination has a left outer flap that comprises a thin leaf of resiliently flexible, biocompatible material, and a right outer flap also comprising a thin leaf of resiliently flexible, biocompatible material. As used herein, a leaf of material should not be interpreted to require a consistent thickness, nor should it be interpreted to require a varying thickness or any other property or structure not expressly set forth. A left nasal prong aperture is disposed through the left outer flap, the aperture configured for closely receiving the left nasal prong of the nasal cannula therethrough. Similarly, a right nasal prong aperture is disposed through the right outer flap, that aperture configured for closely receiving the right nasal prong therethrough. Under this construction, gas provided by the tubing can be inhaled through the left and right nasal prongs. During inhalation, the left and right outer flaps of the spring lamina valve will tend to be deflected toward the left and right nares of the individual to prevent unintended oxygen entrainment. During exhalation, the left and right outer flaps of the spring lamina valve will tend to be deflected away from the left and right nares of the individual to permit exhalation not only through but also around the nasal prongs.

In certain embodiments, the left outer flap and the right outer flap comprise separate members. In other embodiments, the left outer flap and the right outer flap of the spring lamina valve are connected by a bridge portion. When the left and right nasal prongs are received through the left nasal prong aperture and the right nasal prong aperture respectively, the spring lamina valve has a resting contour when not deflected by inspiration or expiration, and the spring lamina valve has a peripheral shape with a left distal end portion, a right distal end portion, and an inner edge for being disposed adjacent to the upper lip and philtrum of the individual. The left outer flap, the right outer flap, and the bridge portion can, for insance, be unitarily formed. The inner edge can be indented to pursue a generally sinusoidal shaped indentation centered around the bridge portion configured to accommodate the upper lip and philtrum of the individual. In embodiments of the spring lamina valve, the resting contour when the left and right nasal prongs are received through the left nasal prong aperture and the right nasal prong aperture respectively has an upturned left distal end portion, an upturned right distal end portion, and a lower central portion coincident with the bridge portion thereby corresponding to the shape of the end portions of the nares and columella of a typical nose.

In practices of the invention, the spring lamina valve further comprises a left inner flap resiliently and hingedly coupled to the left outer flap along the left nasal prong aperture by a left flap isthmus and a right inner flap resiliently and hingedly coupled to the right outer flap along the right nasal prong aperture by a right flap isthmus. Under this construction, when the left and right nasal prongs are respectively received through the left and right apertures, the left and right inner flaps will tend to bow the left and right outer flaps by the resilient force exerted by the inner flaps and the connecting isthmuses.

Where the left and right nasal prong apertures are round, each of the left and right flap isthmuses can span an arcuate angular range. Moreover, the left inner flap is preferably resiliently and hingedly coupled to the left outer flap by the left flap isthmus along a laterally outer portion of the left nasal prong aperture, and the right inner flap is preferably resiliently and hingedly coupled to the right outer flap by the right flap isthmus along a laterally outer portion of the right nasal prong aperture. With this the left and right lateral end portions of the spring lamina valve will tend to be bowed by the force of the inner flaps and the connecting isthmuses.

As disclosed herein, the left outer flap, the left inner flap, the left flap isthmus, the right outer flap, the right inner flap, the right flap isthmus, and the bridge portion can be formed from a single panel of resilient material with the left and right nasal prong apertures formed therethrough with incomplete formation of the apertures leaving the connecting isthmuses and the hanging inner flaps. Still more particularly, the single panel of resilient material can in certain practices be formed from a resilient polymeric material with a thickness of between approximately 0.05 mm and approximately 2.00 mm. Spring lamina valves can be coded based on expiratory resistance provided by the spring lamina valve, and spring lamina valves can be provided in kits with spring lamina valves of different expiratory resistances.

Where the left outer flap and the right outer flap comprise separate members, a first left inner flap can be resiliently and hingedly coupled to the left outer flap along the left nasal prong aperture by a first left flap isthmus, and a first right inner flap can be resiliently and hingedly coupled to the right outer flap along the right nasal prong aperture by a first right flap isthmus. With this, when the left and right nasal prongs are respectively received through the left and right apertures, the first left and right inner flaps will tend to bow the left and right outer flaps in correspondence to the shape of a nare of a patient.

Still further, where the left outer flap and the right outer flap comprise separate members, a second left inner flap can be resiliently and hingedly coupled to the left outer flap along the left nasal prong aperture by a second left flap isthmus, and a second right inner flap can be resiliently and hingedly coupled to the right outer flap along the right nasal prong aperture by a second right flap isthmus. Then, when the left and right nasal prongs are respectively received through the left and right apertures, the second left and right inner flaps will each tend to bow the left and right outer flaps. In particular embodiments, the first and second left flap isthmuses are disposed in general opposition, and the first and second right flap isthmuses are disposed in general opposition thereby tending to cooperate in bowing the left and right outer flaps into bowl or frusto-conical shapes.

While the inner flaps and the isthmuses connecting the same to the outer flaps establish an elegant compliant biasing structure for resiliently biasing the left and right outer flaps of the spring lamina valve away from the nasal cannula body and into a dymanic sealing engagement with the nares of the patient, other biasing structures are readily possible within the scope of the invention. For instance, in one alternative embodiment, the compliant biasing structure comprises a plurality of springs configured to be interposed between the left and right outer flaps of the spring lamina valve and the nasal cannula body. In another embodiment, the compliant biasing structure comprises at least one resiliently compressible foam member configured to be interposed between the left and right outer flaps of the spring lamina valve and the nasal cannula body. Still further, protuberating formations, such as annular ribs, can be disposed on the left and right nasal prongs of the nasal cannula so that the spring lamina valve can be stably held in position. In still another embodiment, the compliant biasing structure takes the form of pairs of repelling magnets, each with a first magnet retained by the nasal cannula and a second magnet retained by the spring lamina valve. In a further alternative, the compliant biasing structure can take the form of a biasing balloon configured for being interposed between the spring lamina valve and the nasal cannula. It is possible within the scope of the invention for a wall of that biasing balloon to form the left outer flap and the right outer flap of the spring lamina valve. Yet further, the compliant biasing structure can comprise at least one resiliently compressible tubular member configured to be interposed between the left and right outer flaps of the spring lamina valve and the nasal cannula body. For instance, plural tubular or semi-tubular members can be interposed between the spring lamina valve and the nasal cannula body. Each such compliant biasing structure advantageously promotes sealing engagement of the spring lamina valve with the nares of the patient during inhalation while permitting exhalation through and around the nasal prongs of the cannula during exhalation.

One will appreciate that the foregoing discussion broadly outlines the more important goals and features of the invention to enable a better understanding of the detailed description that follows and to instill a better appreciation of the inventors’ contribution to the art. Before any particular embodiment or aspect thereof is explained in detail, it must be made clear that the following details of construction and illustrations of inventive concepts are mere examples of the many possible manifestations of the invention.

### BRIEF DESCRIPTION OF THE DRAWINGS

In the accompanying drawing figures:

FIG. 1 is a schematic view of an oxygen dispensing and conservation system according to the present invention;

FIG. 2 is a forward perspective view of the housing of an oxygen dispensing and conservation system as taught herein;

FIG. 3 is a rearward perspective view of the housing of the oxygen dispensing and conservation system;

FIG. 4 is a sectioned view in side elevation of an oxygen dispensing and conservation system pursuant to the invention;

FIG. 5 is a rearward perspective view of the oxygen dispensing and conservation system with the top, bottom, and sidewalls of the housing removed;

FIG. 6 is a perspective view of a nasal cannula adapted for use with the oxygen dispensing and conservation system to permit expiration through the nasal cannula and direct and immediate control over the fraction of inspired oxygen (Fi02) inhaled through the nasal cannula;

FIG. 7 is an alternative perspective view of the nasal cannula of FIG. 6;

FIG. 8 is a bottom plan view of the nasal cannula of FIG. 6;

FIG. 9 is a perspective view of an alternative embodiment of the nasal cannula of the present invention;

FIG. 10 is an exploded view of the nasal cannula of FIG. 9;

FIG. 11 is a lower perspective view of the nasal cannula of FIG. 9;

FIG. 12 is another exploded view of the nasal cannula of FIG. 9;

FIG. 13 is a schematic view of the system in operation during a series of respiratory cycles;

FIG. 14 is a perspective view of an oxygen dispensing and conservation system according to the invention with a nasal cannula incorporating a spring lamina valve as taught herein;

FIG. 15 is a perspective view of a spring lamina valve pursuant to the invention;

FIG. 16 is a top plan view of the spring lamina valve;

FIG. 17 is a perspective view of a nasal cannula incorporating a spring lamina valve as disclosed herein;

FIG. 18 is a perspective view of the nasal cannula and spring lamina valve of FIG. 17 with the spring lamina valve resiliently deflected;

FIG. 19 is a perspective view of the nasal cannula and spring lamina valve during application to the nose of a patient;

FIG. 20 is a perspective view of the nasal cannula and spring lamina valve in situ relative to the nose of the patient;

FIG. 21 is a front elevation view of the nasal cannula and spring lamina valve in situ relative to the nose of the patient during inhalation with the spring lamina valve and the lower portion of the nose sectioned;

FIG. 22 is a front elevation view of the nasal cannula and spring lamina valve in situ relative to the nose of the patient during exhalation with the spring lamina valve and the lower portion of the nose sectioned;

FIG. 23 is a perspective view of an alternative embodiment of the spring lamina valve and a nasal cannula incorporating the same;

FIG. 24 is a partially sectioned view of another alternative embodiment of the spring lamina valve and a nasal cannula incorporating the same;

FIG. 25 is a partially sectioned view of a spring lamina valve with biasing coil springs and a nasal cannula incorporating the same;

FIG. 26 is a top plan view of the spring lamina valve with biasing coil springs of FIG. 25;

FIG. 27 is a partially sectioned view of a spring lamina valve with biasing bellows springs and a nasal cannula incorporating the same;

FIG. 28 is a top plan view of the spring lamina valve with biasing bellow springs of FIG. 27;

FIG. 29 is a partially sectioned view of a spring lamina valve with biasing resiliently compressible members and a nasal cannula incorporating the same;

FIG. 30 is a top plan view of the spring lamina valve with biasing compressible members of FIG. 29;

FIG. 31 is a partially sectioned view of a spring lamina valve with biasing ribs and a nasal cannula incorporating the same;

FIG. 32 is a partially sectioned view of a spring lamina valve with biasing repelling magnets and a nasal cannula incorporating the same;

FIG. 33 is a partially sectioned view of a spring lamina valve with a biasing inflatable body and a nasal cannula incorporating the same;

FIG. 34 is a partially sectioned view of a spring lamina valve with biasing resilient columns and a nasal cannula incorporating the same;

FIG. 35 is a partially sectioned view of a spring lamina valve with biasing resilient tubes and a nasal cannula incorporating the same; and

FIG. 36 is a partially sectioned view of a spring lamina valve with biasing resilient arcuate members and a nasal cannula incorporating the same.

### DETAILED DESCRIPTION OF PREFERRED EMBODIMENTS

The spring lamina valve, the nasal cannula, and the ambient-pressure oxygen dispensing and conservation system with which they are operative are subject to a wide variety of embodiments. However, to ensure that one skilled in the art will be able to understand and, in appropriate cases, practice the inventions disclosed herein, certain preferred embodiments of the broader inventions are described below and shown in the accompanying drawing figures.

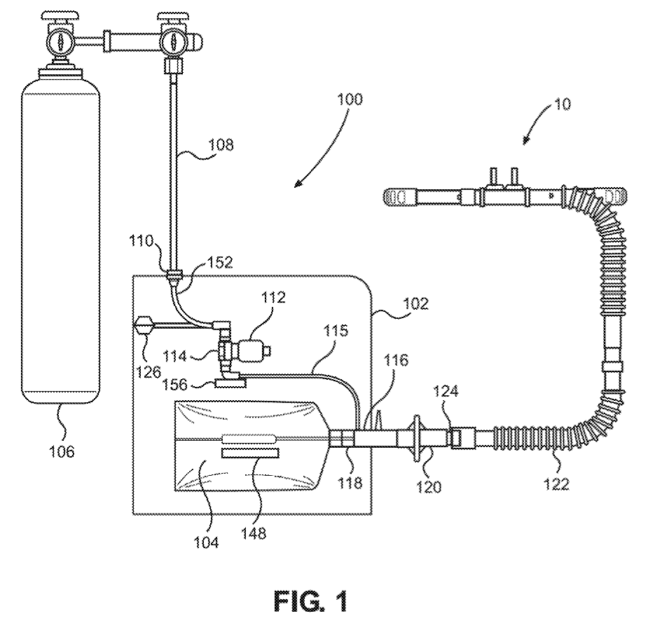

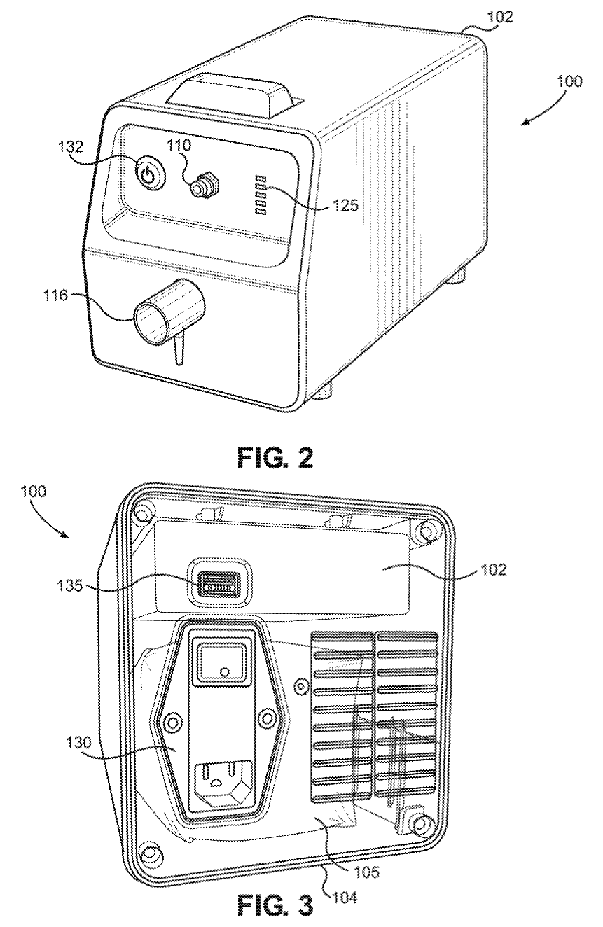

To understand and appreciate the utility and operation of the nasal cannula 10 and spring lamina valve 50 for a nasal cannula 10 disclosed herein, reference will first be had to the oxygen dispensing and conservation system 100 relative to which they are designed to function. With reference to the schematic view of FIG. 1 and to the depictions of the oxygen dispensing and conservation system 100 of FIGS. 2-5, the oxygen dispensing and conservation system 100 provides an on-demand supply of oxygen at ambient pressure to a recipient, in this case through the nasal cannula 10, from a donor reservoir 104. The donor reservoir 104 retains oxygen at ambient pressure and is continually and automatically supplied with oxygen from an oxygen source 106, such as a tank of compressed oxygen gas or liquid oxygen.

With the donor reservoir 104 retaining oxygen at ambient pressure and with the donor reservoir 104 being automatically replenished, a full and ample supply of oxygen is constantly available for patient inspiration. Since oxygen is withdrawn from the reservoir 104 only through inspiration, oxygen losses during patient expiration are substantially eliminated. The supply of oxygen is thus conserved without compromising availability to the individual recipient.

The donor reservoir 104 in this embodiment comprises an expandable and compressible shell, bladder, or other expandable and compressible body that is disposed within a housing 102. The housing 102 could be a primary housing or a sub-housing within a larger structure. However, except as the claims may expressly require, the donor reservoir 104 need not necessarily be within a housing 102 to be within the scope of the invention. The housing 102 in the depicted embodiment defines an enveloping boundary for the reservoir 104 so that the shell of the reservoir 104 presses toward one or more portions of the boundary defined by the housing 102 as the reservoir 104 is expanded. In the depicted, non-limiting example, the housing 102 has a bottom that defines a lower boundary for the reservoir 104, a top that defines an upper boundary for the reservoir 104, and distal ends that define longitudinal boundaries for the reservoir 104.

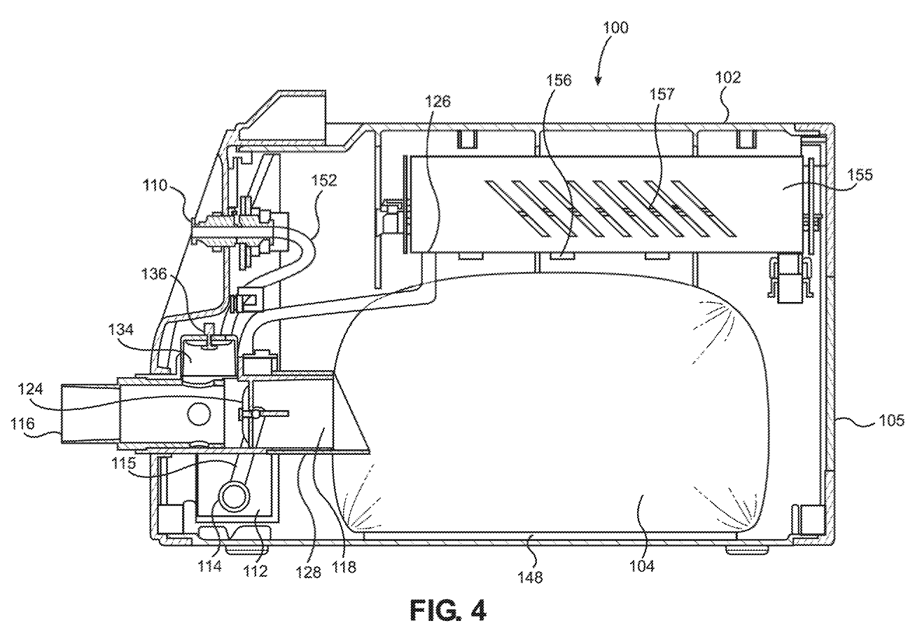

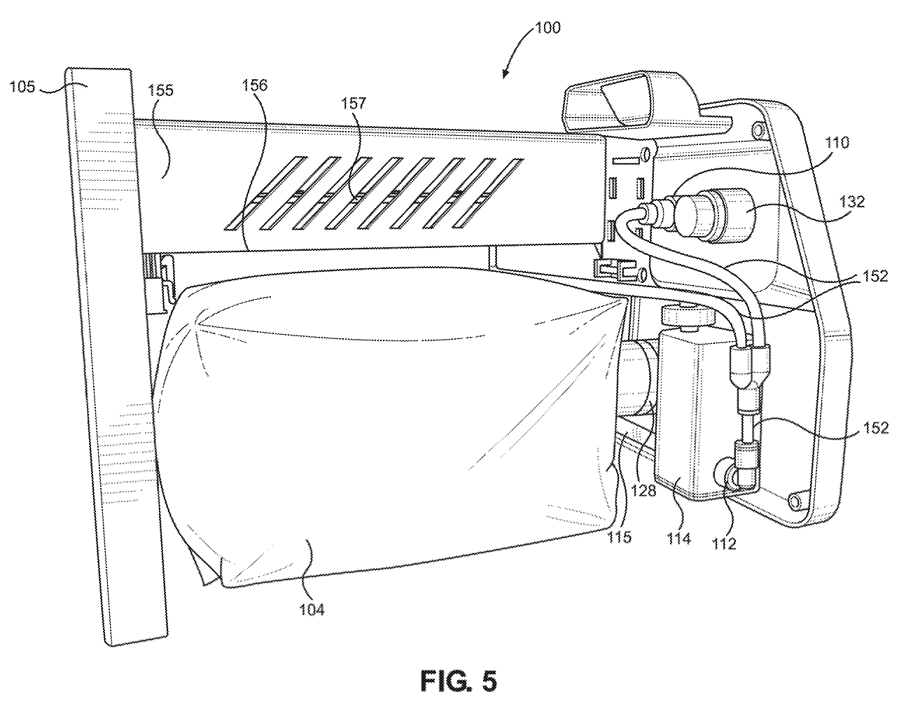

As shown in FIGS. 4 and 5, the housing 102 in this example defines an elongate, generally cubic inner volume, and the reservoir 104 has a corresponding elongate, generally cubic shape. Other shapes and combinations of shapes are readily possible and within the scope of the invention except as it might be expressly limited by the claims. In certain practices of the invention, the lower wall portion of the shell of the reservoir 104 can be adhered or otherwise secured to the housing 102 in one or more locations, such as by an adhesive strip 148 as depicted in FIG. 4 or in any other manner. In this example, the reservoir 104 has four elongate sidewalls joined to exhibit a rectangular cross section when inflated, a first end wall formed by four triangular portions that extend from the first ends of the sidewalls and joined, and a second end wall likewise formed by four triangular portions that extend from the second ends of the sidewalls and joined. The edges of the walls are joined in a sealed manner to define the cubic reservoir 104. The reservoir 104 is thus sealed but for an entry orifice at the first end of the reservoir 104.

The shell of the reservoir 104 is formed from a flexible and substantially gas impermeable material. One of ordinary skill in the art would be aware of numerous such materials. Each is within the scope of the invention except as expressly excluded by the claims. The shell of the reservoir 104 could, for example, be formed from a flexible polymeric material with or without a lining layer. The material defining the reservoir 104 could comprise a foil formed by one or more layers of polymeric material with an aluminum lining. The reservoir 104 can have combinations including one or more flexible walls, rigid walls, compressible walls, collapsible walls, expandable walls, thin walls, or other walls capable of keeping a volume of gas inside. Other formations of the reservoir 104 are possible and within the scope of the invention.

Preferably, as is enabled by formation of the reservoir 104 of a lightweight, flexible foil, the reservoir 104 once expanded tends to substantially maintain an expanded shape and configuration, whether by its own structural integrity or otherwise, even when it is open to ambient pressure, such as by a fluidic connection to the recipient. The fluidic connection to the recipient can be achieved, for example, through ambient pressure tubing 122 and, ultimately, through a patient delivery device, such as the nasal cannula 10 disclosed herein, through a breathing mask, or through another mechanism for conveying oxygen to a recipient. Since the system 100 is designed to provide oxygen on demand during nature inspiration by the patient, the ambient pressure tubing 122 has a large inner diameter to reduce any resistance of patient inhalation to near zero. As taught herein, once expanded, the reservoir 104 in preferred embodiments does not significantly collapse on its own due to the weight of its walls. When filled with oxygen, the reservoir 104 thus temporarily stores a compartmented volume of oxygen at ambient pressure waiting to be drawn therefrom by the recipient.

In the embodiment of the oxygen dispensing and conservation system 100 of FIG. 1, a fluidic connector 118, which may be considered a T-connector, has a first, longitudinal port in fluidic communication with the donor reservoir 104, such as through a tubular connector 128 that is fixed and sealed within the aperture in the neck of the reservoir 104. The fluidic connector 118 has a second, longitudinal port comprising an output connector 116 in fluidic communication with the ambient pressure tubing 122 and, through that tubing 122, the recipient, such as through the nasal cannula 10, a breathing mask, or another gas delivery mechanism. Finally, the fluidic connector 118 has a third, lateral port between the first and second openings in fluidic communication with the oxygen source 106 in this case through a flow-limiting connector 115. The first, second, and third ports are in fluidic communication with one another within the fluidic connector 118. The fluidic communication from the source 106 to the connector 118 could, for instance, be through high-pressure tubing 108 from the oxygen source 106 to an oxygen connector 110 fixed to the housing 102 and high-pressure tubing 152 from the oxygen connector 110 to a supply valve 112. A pressure sensor 126 is interposed to detect gas pressure entering the supply valve 112, such as by being fluidically interposed along the high-pressure tubing 152.

Further, in the embodiment of the invention depicted in FIGS. 4 and 5, ambient air can be selectively entrained with oxygen drawn from the donor reservoir 104 by operation of a gas blending device formed by the fluidic connector 118. There, one can perceive that a one-way inspiratory valve 124 is disposed within the fluidic connector 118 between the first port of the connector 118 in communication with the reservoir 104 and the second port of the connector 118 in communication with the output connector 116. The flow-limiting connector 115 is disposed proximal to the one-way inspiratory valve 124 with respect to the donor reservoir 104, and an air-input connector 134 is disposed distal to the one-way inspiratory valve 124 with respect to the donor reservoir 104 and thus proximal to the output connector 116 with respect to the one-way inspiratory valve 124. Further, a one-way air input valve 136 is fitted to the air-input connector 134. Under this configuration, the donor reservoir 104 can be replenished with oxygen or potentially another gas through the flow-limiting connector 115, and the oxygen or other gas within the donor reservoir 104 can be drawn therefrom during inspiration through the output connector 116 with ambient air being selectively entrained therewith through the air-input connector 134. During expiration, expired gas is prevented from entering the donor reservoir 104 by the one-way inspiratory valve 124.

The supply valve 112, which in this example comprises an electromechanical solenoid valve 112, has an open condition and a closed condition. The valve 112 is fluidically interposed between the pressurized oxygen source 106 and the reservoir 104. When the supply valve 112 is in the open condition, oxygen can be passed from the oxygen source 106, through the tubing 108, through the valve 112, through the connector 118, and into the reservoir 104. When the valve 112 is in the closed condition, the passage of oxygen between the oxygen source 106 and the reservoir 104 is prevented.

The one-way inspiratory valve 124 is fluidically interposed between the reservoir 104 and the recipient, such as by being fluidically connected to the second port of the fluidic connector 118 directly. A gas filter 120 is disposed along the path of flow between the reservoir 104 and the cannula 10, such as by being fluidically interposed between the fluidic connector 118 and the ambient pressure tubing 122 as in the example of FIG. 1. With the fluidic connector 118 fluidically connected through its first port to the neck of the reservoir 104, the one-way inspiratory valve 124 is operative to enable gas to flow from the donor reservoir 104, through the ambient pressure tubing 122, and to the recipient, such as through the nasal cannula 10 or a breathing mask, but to prevent reverse gas flow, such as from the recipient and into the donor reservoir 104.

The volume of gas, in this example oxygen, in the donor reservoir 104 is retained substantially at ambient pressure. Ambient pressure can be defined as the pressure of the air surrounding the donor reservoir 104. Substantially at ambient pressure may be understood to be equal to or within any deviation that does not meaningfully affect the function, performance, or intended use of the donor reservoir 104 and the system 100 in general. Where the foregoing is considered to be insufficiently clear, “substantially” should be considered to be any deviation that is within 10% of the ambient pressure external to the reservoir 104. As a recipient undergoes the inspiratory phase of breathing, oxygen will be drawn from the donor reservoir 104 through the ambient pressure tubing 122 thereby drawing from and tending to reduce the volume of oxygen in the donor reservoir 104. Due to the compressible nature of the donor reservoir 104, the reservoir 104 will tend to contract. When it does contract, the donor reservoir 104 is automatically replenished with oxygen or, potentially, another gas. In the present embodiment, inflation of the donor reservoir 104 is triggered by an inflation detection system that detects when the donor reservoir 104 is not fully inflated or is inflated below a predetermined threshold, and that inflation detection actuates the supply valve 112 to an open condition to inflate the donor reservoir 104 while avoiding pressurization of the reservoir 104 so that the oxygen within the reservoir 104 remains substantially at ambient pressure.

The inflation detection system has a first condition wherein replenishing oxygen is not supplied to the donor reservoir 104 and a second condition wherein replenishing oxygen is supplied to the donor reservoir 104. The first condition can be a condition wherein the donor reservoir 104 is inflated with oxygen to a certain, predetermined state of inflation, and the second condition can be a condition wherein the donor reservoir 104 is inflated with oxygen below the predetermined state of inflation. The inflation detection system is operative to detect when the donor reservoir 104 has reached the predetermined state of inflation. The predetermined state of inflation can be detected when the donor reservoir 104 reaches a predetermined size or other inflation condition in any dimension or combination of dimensions. In embodiments of the invention, the donor reservoir 104 can be considered to have a fully inflated condition, and the inflation detection system detects when the donor reservoir 104 is inflated to the fully inflated condition or to within a predetermined range of the fully inflated condition. By way of example and not limitation, the inflation detection system can detect when the donor reservoir 104 is inflated with oxygen at or above a threshold inflation level, which may be equal to or less than the fully inflated condition.

Made aware of the present invention, one skilled in the art may appreciate plural mechanisms that would operate as inflation detection systems to detect when the donor reservoir 104 is inflated to the predetermined state of inflation. Each such mechanism is within the scope of the invention except as it may be expressly limited by the claims. Inflation detection mechanisms could comprise mechanical systems, electrical systems, electromagnetic systems, optical systems, electro-mechanical systems, sound-activated systems, movement sensors, light sensors, and any other type of system effective to detect when the donor reservoir 104 is inflated to a predetermined state of inflation with it again being noted that the predetermined state of inflation may be reached while the oxygen within the donor reservoir 104 is substantially at ambient pressure.

In the embodiments of FIGS. 1 through 5, the inflation detection system takes the form of a contactless detection system 156, which can be an optical detection system 156. The detection system 156 could be carried forth by, for instance, a laser detection system, a camera system, an infrared inflation detection system, or any other effective contactless detection system 156. In certain embodiments, a contactless detection system 156 can be formed with a light emitter, such as a laser or other light emitter, retained to one side of the reservoir 104 and a light receptor disposed to the opposite side of the reservoir 104. Under such constructions, the inflation condition of the donor reservoir 104 can be sensed in a contactless manner, such as where the donor reservoir 104 is inflated to a condition where the reservoir 104 prevents the communication of light from the light emitter to the light receptor, where the reservoir 104 demonstrates a predetermined reflectance value, or in some other contactless manner.

In other embodiments, the detection system 156 comprises one or more proximity sensors that are operative to detect the proximity of the localized, facing surface of the donor reservoir 104. For instance, as in FIGS. 4 and 5, a series of proximity sensors forming the inflation detection system 156 are retained relative to the interior of the housing 102, in this example, relative to an electronics casing 155 that is fixed within the housing 102 above the donor reservoir 104. The electronics casing 155 retains an electronic control system 157 with an electronic circuit board with electronic memory retaining system software, one or more computer processors for processing the software, and further electronic circuitry and wiring necessary for operation of the system 100.

The inflation detection system 156 can thus detect the inflation condition of the donor reservoir 104, potentially at multiple locations therealong. The inflation detection system 156 can detect when the donor reservoir 104 is filled to a predetermined state of inflation. The predetermined state of inflation can be sensed, for example, based on the sensed position of the wall of the donor reservoir 104, such as by a detection of the proximity of the wall of the donor reservoir 104 to the proximity sensors of the inflation detection system 156 or the sensed inflation of the reservoir 104 to obstruct optical communication between an emitter and a receptor.

Based on the state of inflation of the reservoir 104 as detected by the inflation detection system 156, the flow switch 114 is operative to actuate the valve 112 between the ON condition where oxygen is permitted to flow from the oxygen source 106 to the donor reservoir 104 and the OFF condition where such flow is prevented. More particularly, when the donor reservoir 104 is detected by the inflation detection system 156 to be below the predetermined state of inflation based on the inward contractive movement of the walls of the donor reservoir 104, the flow switch 114 will trigger the valve 112 to the ON condition to permit oxygen to flow from the oxygen source 106 to fill the donor reservoir 104. When the donor reservoir 104 reaches the predetermined state of inflation based on a detected expansion of the walls of the donor reservoir 104, again as detected by the inflation detection system 156, the flow switch 114 will trigger the valve 112 to the OFF condition to prevent the further flow of oxygen beyond the predetermined state of inflation thereby preventing pressurization of the donor reservoir 104 and preventing expelling oxygen or other gas from the system 100. The flow switch 114 thus has an activated state, which may be considered to be the ON condition, when the donor reservoir 104 is detected to be below the predetermined state of inflation, and flow switch 114 has a deactivated state, which may be considered to be the OFF condition, when the donor reservoir 104 is detected to have reached the predetermined state of inflation based on a detection by the inflation detection system 156 of the expansion of the donor reservoir 104.

In another non-limiting embodiment, the inflation detection system comprises an electro-mechanical system for detecting when the donor reservoir 104 is filled to the predetermined state of inflation. Such an inflation detection system (not illustrated in the present embodiment) can have a contact structure disposed to contact, to be contacted by, to be moved by, or otherwise to be actuated by the donor reservoir 104 when the reservoir 104 reaches a stage of inflation. Within the scope of the invention, the location and construction of the contact structure could vary. The contact structure can, for instance, be disposed to project from or through the distal end wall of the housing 102 and into the inner volume of the housing 102 so that it projects toward and can engage the distal end of the reservoir 104. In other embodiments, the contact structure can be disposed to project from or through the upper wall of the housing 102 and into the inner volume of the housing 102 to engage a mid-portion of the reservoir 104. The contact structure can, for example, be retained by a support structure fixed to the upper wall or another upper portion of the housing 102.

The contact structure is positioned to be moved by the donor reservoir 104 as the reservoir 104 expands toward an inflated condition. The contact structure can, for instance, be depressed, pivoted, rotated, or otherwise actuated by the donor reservoir 104 and more particularly by an expansion of the donor reservoir 104. The contact structure can then operate as or as a component of or to actuate a flow switch 114. When the contact structure is actuated by the expansion of the donor reservoir 104, the flow switch 114 is operative to actuate the valve 112 between the ON condition where oxygen is permitted to flow from the oxygen source 106 to the reservoir 104 to replenish and fill the reservoir 104 and the OFF condition where oxygen is prevented from flowing from the oxygen source 106 to the reservoir 104. The contact structure can be biased, such as by spring force, under the force of gravity, by resiliency, or any other biasing method or combination thereof toward the donor reservoir 104.

In the non-limiting embodiment of FIGS. 1-5, the donor reservoir 104 is disposed within a housing 102. Additionally or alternatively, the donor reservoir 104 could be disposed within a sub-housing that, in turn, could be disposed in the housing 102 or that could stand independently. Still further, the donor reservoir 104 could be disposed without a housing or enclosure, in which case the contact structure and potentially the flow switch could be otherwise retained, such as by a surrounding band, a rigid arm, or another retaining structure, for contact or other sensing or engagement relative to the donor reservoir 104. The contact structure and the flow switch 114 could be retained together, potentially as a unit, or in separate locations.

The contact structure is permitted to move inwardly in the direction toward the donor reservoir 104 when the volume of oxygen in the donor reservoir 104 falls below a predetermined state of inflation such that the outside wall is, or can be, deflected or moved inwardly. The flow switch 114 has an activated state, which may be considered to be the ON condition, when the contact structure is sufficiently moved, such as by extension, pivoting, or other movement, in an inward direction toward the inner volume of the donor reservoir 104. The flow switch 114 has a deactivated state, which may be considered to be the OFF condition, when the contact structure is moved, such as by retraction, pivoting, or other movement in an outward direction away from the donor reservoir 104. The contact structure is moved outwardly to adjust the flow switch 114 to the deactivated state, which is the OFF condition, when the volume of oxygen in the donor reservoir 104 reaches the predetermined state of inflation to cause the outside wall of the donor reservoir 104 to be advanced outwardly by the expansion of the donor reservoir 104. For instance, where the contact structure is a depression switch, expansion of the donor reservoir 104 will press the outer wall or shell of the donor reservoir 104 outwardly to press the contact structure and the flow switch 114 to the deactivated state.

In embodiments of the oxygen dispensing and conservation system 100, the supply valve 112 can comprise a solenoid valve that is in electrical communication, such as through electrical wiring in an electrical circuit, with the flow switch 114. As illustrated, an electrical control system 157, which can include electrical circuitry, electronic memory, wiring, system software retained and operative by electrical circuitry and electronic memory, and other electrical control and connection components, cooperates with the inflation detection system to induce the solenoid supply valve 112 to an open condition to permit the flow of oxygen from the source 106 when the flow switch 114 is in the activated state. The electrical control system can receive power from a power source, which could be a source of alternating current through a power supply connection 130, a source of direct current such as a battery power source, or some other source of electric power. The flow of electrical power from the power source can be controlled by a power switch 132. The solenoid valve 112 is induced by the inflation detection system and the electrical control system to a closed condition to prevent the flow of oxygen from the source 106 to the reservoir 104 when the flow switch 114 is in the deactivated state. Each of the components referenced herein can be further combined or separated within the scope of the invention.

Even when the valve 112 is in an open condition, the rate of flow, the pressure of flow, or both the pressure and rate of flow of oxygen from the source 106 to the donor reservoir 104 are limited by the flow-limiting connector 115. The flow-limiting connector 115 could limit the flow rate of oxygen from the source 106 to the donor reservoir 104 to a predetermined flow rate, such as less than 1 liter per minute or any other flow rate. The flow-limiting connector 115 could, for example, comprise a narrow-diameter tube connector, such as a connector having an inner diameter of 0.02 mm or some other dimension reduced as compared to other conduit connections within the fluidic system. Rapid changes in pressure within the donor reservoir 104 can thus be prevented on opening of the valve 112.

While a compressed gas tank is often depicted and referred to as the oxygen source 106 herein, other oxygen sources 106 are possible within the scope of the invention. By way of further, non-limiting examples, the system 100 can provide on-demand oxygen to patients with oxygen supplied by an oxygen concentrator, which takes in air and removes the nitrogen from it thereby leaving the oxygen-enriched gas for those patients requiring medical oxygen. The typical flow of this compressed oxygen is 1-5 liters /minute. High-end oxygen concentrators can deliver upwards of 50L/minute, but they require more electricity and more maintenance.

Where the oxygen source 106 is an oxygen concentrator, the system 100 can be placed between the oxygen concentrator and the oxygen delivery device so that, as oxygen leaves the concentrator, it enters the large reservoir 104 where it remains at ambient pressure until the patient inhales. As the patient breathes in and draws oxygen from the reservoir 104, the reservoir 104 begins to deplete, the supply valve 112 from the oxygen concentrator as the oxygen source 106 opens to replenish the reservoir 104 with compressed oxygen from the oxygen concentrator. As the patient exhales, no flow occurs between the reservoir 104 and the patient. Rather than wasting the oxygen flowing from the concentrator during the exhalation phase of the patient, the flow is employed to replenish the reservoir 104. Once the reservoir 104 is full, the supply valve 112 stops the flow of oxygen from the oxygen source 106. The cycle can repeat with every breath. In this manner, oxygen not taken in by the patient during inspiration is stored rather than lost.

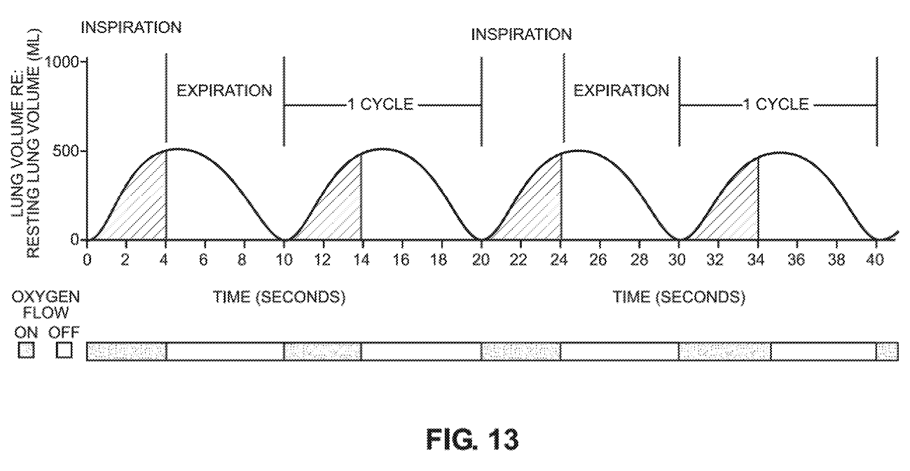

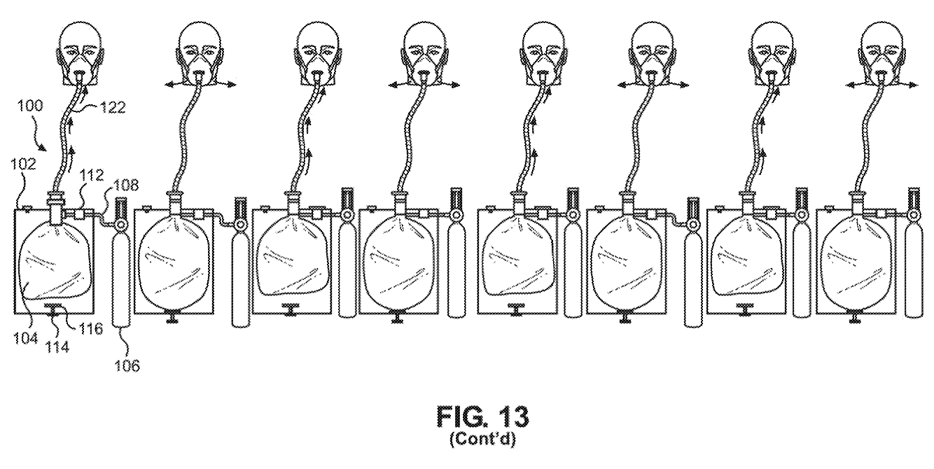

Referring to FIG. 13, the system 100 is depicted in operation during a series of respiratory cycles to provide an on-demand supply to a recipient, such as through a mask or the nasal cannula 10 disclosed herein worn by a patient in need. In operation of the system 100, inspiration by the patient will operate to draw oxygen at ambient pressure from the donor reservoir 104 thereby tending to contract the reservoir 104. When the reservoir 104 falls below the predetermined state of inflation, oxygen is allowed to flow and the reservoir 104 is automatically filled to the predetermined state of inflation. A volume of continually-replenished oxygen at ambient pressure is thus available within the reservoir 104 to be drawn through the ambient pressure tubing 122 during a natural inspiration phase of a breathing cycle. When the recipient is not engaged in inspiration, no oxygen is drawn and no oxygen tends to be expelled from the reservoir 104. When the volume of oxygen within the reservoir 104 falls below the predetermined state of inflation, the inflation detection system will detect the same and trigger the valve 112 to an open condition. Flow of oxygen is then permitted from the oxygen source 106 so that the donor reservoir 104 will be filled with oxygen until the predetermined state of inflation is reached.

When the predetermined state of inflation is reached, the inflation detection system will detect the same and trigger the valve 112 to a closed condition to prevent the further supply of oxygen to the donor reservoir 104 from the source 106 until a further inspiration phase of a breathing cycle draws a volume of oxygen from the reservoir 104. The donor reservoir 104 is thus automatically supplied with oxygen while pressurization of the oxygen in the reservoir 104 above ambient pressure is automatically prevented. Supplemental oxygen is safely and effectively supplied to the patient at ambient pressure in an on-demand volumetric displacement system enabling the transfer of oxygen during the entire inspiratory phase of the breathing cycle while the wasteful release of oxygen to the atmosphere is prevented, including during the expiratory phase.

The donor reservoir 104 thus automatically receives replenishing oxygen from the pressurized source 106 through the high-pressure tubing 108 and through the supply valve 112. The automatic refilling of the reservoir 104 ensures that the donor reservoir 104 always retains a supply of oxygen available for the next inspiratory phase of the breathing cycle while the oxygen in the reservoir 104 never exceeds ambient pressure.

Where the donor reservoir 104 is visually exposed, such as through a partially or completely transparent housing 102 or an observation aperture or window in the housing 102, an observer is provided with visual confirmation of the state of inflation of the donor reservoir 104. For example, as FIG. 3 illustrates, the housing 102 of the depicted embodiment has a translucent rear wall portion 105 that permits one to observe the inflation condition of the donor reservoir 104, which can be of useful comfort in confirming proper operation in view of the quiet operation of the ambient-pressure oxygen dispensing and conservation system 100. Further, the system 100 includes an electronic status indicator 125 as is shown in FIG. 2 to confirm the state of inflation of the donor reservoir 104. The electronic status indicator 125 can, for example, comprise a series of light sources or another visual electronic indicator of the level to which the donor reservoir 104 is filled. The system 100 can thus provide a synchronized delivery of supplemental oxygen to a recipient as the donor reservoir 104 and the system 100 in general synchronize with the physiological ventilations of a patient based on the storage and replenishment of oxygen in the donor reservoir 104 at ambient pressure and the termination of the supply of oxygen automatically when the donor reservoir 104 reaches the predetermined state of inflation.

Within the scope of the invention, the system 100 can measure, record, and analyze the flow of oxygen and the breathing characteristics of a patient, including by use of the electrical control system 157 and, potentially or alternatively, through remote data communication and data processing by wireless communication. As shown in FIG. 3, a data port 135, such as a USB port or another data port, allows wired communication to and from the electrical control system 157 and the system 100 in general. By wired or wireless communication using the data port 135 or a wireless communication protocol, system software can be updated and modified, the system 100 can be programmed and adapted to particular requirements, and acquired data, such as data regarding system operation, user respiration, and other aspects, can be downloaded for use and analysis.

To facilitate such data acquisition and analysis, a volumetric measuring flow meter could be connected to the source 106 of oxygen. Additionally or alternatively, one or more flow meters could be retained within the housing 102 along the path of gaseous flow through the system 100. For instance, a flow meter could be disposed to measure oxygen passing through the valve 112. The valve 112 can incorporate a flow meter, or a flow meter could be otherwise disposed. A flow meter could further or alternatively be disposed between the reservoir 104 and the ambient pressure tubing 122. By measuring the volume of oxygen supplied to a recipient by the system 100, such as over a given time period, per cycle of inspiration and expiration, or otherwise, plural determinations, measurements, and analyses can be made. For instance, one can determine the volume of oxygen inspired by the patient and, additionally or alternatively, the volume of oxygen remaining in the oxygen source 106. Through electronic memory and software operating on the electrical system or in communication therewith, whether by direct incorporation, wireless communication, or a combination thereof, the system 100 can harvest, process, and analyze data based on usage of the system 100.

By use of the oxygen dispensing and conservation system 100, a patient can naturally draw supplemental oxygen from the donor reservoir 104 through a breathing mask, through a nasal cannula 10 as disclosed herein, or through another delivery apparatus. Alternative recipient delivery apparatuses could, for example, comprise laryngeal mask airways (LMA), endotracheal tubes, tracheostomys, ventilator attachments, CPAP machine connectors, Ambu bags, or even delivery devices for oxygen delivery during recreation.

The on-demand supply of oxygen to be naturally inspired that is provided by the donor reservoir 104 with the present system 100 overcomes numerous deficiencies and limitations exhibited by systems of the prior art. For instance, to achieve the prescribed inspired oxygen concentration, many prior art systems are dependent on the patient's peak inspiratory flow rate (PIFR). For example, when a patient requires a low-inspired oxygen concentration, using a nasal cannula at a low continuous flow rate may help, but this practice limits the patient’s oxygen to a low inspired oxygen concentration only. Should the patient’s oxygen requirements increase significantly, the inspiratory effort to drive more air into the lungs, which is dependent on tidal volume, 'speed' of inspiration, and respiratory rate, will make the PIFR exceed the flow rate at which oxygen or an oxygen/air mixture is supplied by the delivery device. This will mean that at the time of PIFR more or less entrainment of room air occurs, altering the resulting FiO2 in an unpredictable fashion. On the other hand, while using a non-rebreathing face mask at very high flows of oxygen (10-15L/Min) is capable of providing a reliable delivery of oxygen volume at the prescribe concentration while being less dependent on PIFR, large volumes of oxygen are wasted to the environment with oxygen continuing to flow even during expiration.

The system 100 is thus capable of passively permitting the transfer of oxygen or another gas or gases from the ambient pressure reservoir 104 by making the gas or gases available to the recipient in a manner that matches the exact volume and rate of demand by the recipient. The drop in pressure induced by inhalation is used for the transfer of volume from the reservoir 104. No extra pressure is required, such as to open a pressure check valve, to start the flow as may be required where a chamber or reservoir contains oxygen at a higher pressure than ambient pressure.

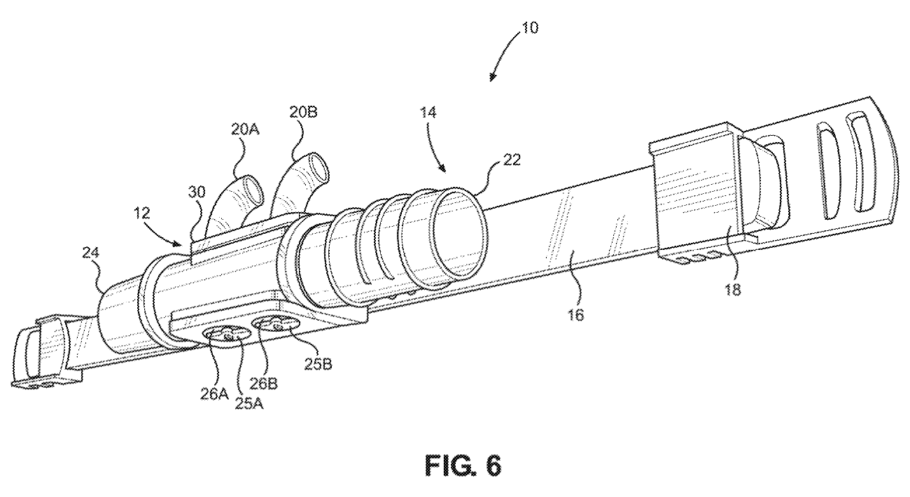

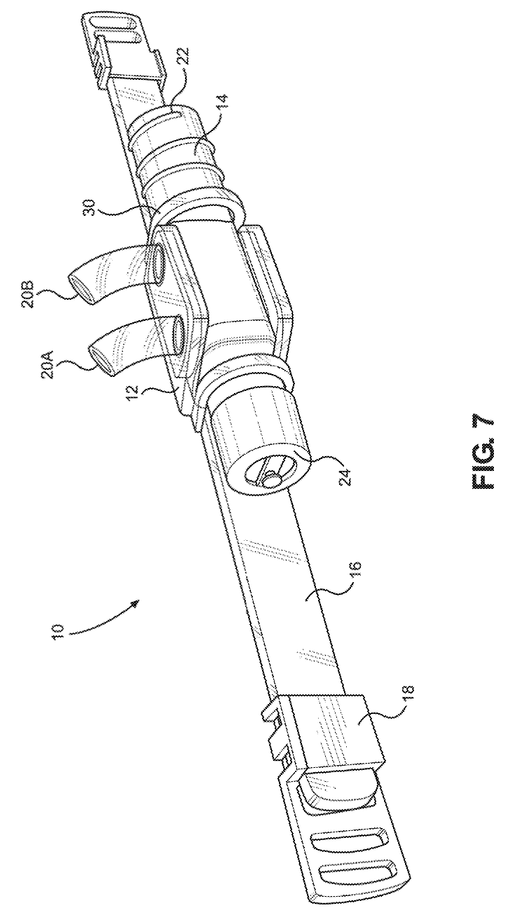

As taught herein, oxygen from the donor reservoir 104 can be made available for inspiration by a recipient through a breathing mask, a nasal cannula 10 as disclosed herein, or another delivery device. An embodiment of a nasal cannula 10 according to the invention is depicted in FIGS. 6-8. There, the nasal cannula 10 is formed with a first body member 12 that is assembled with a second body member 14. In the present embodiment, the first body member 12 is formed from a resilient and relatively flexible material, such as but not limited to silicone rubber or thermoplastic polyurethane while the second body member 14 is formed from a relatively rigid material, such as but again not limited to a rigid plastic. The first body member 12 has a central portion that defines a reception cavity, which can be generally tubular, and the second body member 14, which can likewise be generally tubular, can be received into and retained by the first body member 12 with a central portion of the second body member 14 passing through the reception cavity of the first body member 12. The central portion of the first body member 12 has a nasal prong platform 30, and first and second nasal prongs 20A and 20B extend in parallel from the nasal prong platform 30 for being received into the nares of a patient.

First and second straps 16 extend laterally in opposite directions from the central portion of the first body member 12, and buckles 18 are retained at the distal ends of the straps 16. With that, the nasal cannula 10 can be retained relative to the head of a wearer by the straps 16 and buckles 18, possibly in combination with an additional fastening mechanism, such as a further strap passing behind the user’s neck or head, by cloth tape, or by any other fastening method that would be obvious to one skilled in the art after reviewing the present disclosure.

Within the scope of the invention, the first and second body members 12 and 14 can be interchangeable and replaceable relative to one another to suit particular patient needs and preferences. For instance, first body members 12 with differently sized or spaced nasal prongs 20A and 20B can be provided to be fitted with a second body member 14. By way of non-limiting example, first body members 12 might be provided with nasal prongs 20A and 20B having 4 mm, 5 mm, 6 mm, and 7 mm outer diameters for comfortably being received into and engaging the nares of different users.

The second body member 14 has an open inner volume with an opening or openings disposed to align with the nasal prongs 20A and 20B of the first body member 12 when the body members 12 and 14 are mutually engaged. Further, the central portion of the first body member 12 is open, such as through an opening or openings, generally opposite to the nasal prongs 20A and 20B. The second body member 14 has first and second FiO2 adjustment apertures 25A and 25B and first and second adjustable aperture covers 26A and 26B. The FiO2 adjustment apertures 25A and 25B are in fluidic communication with the inner volume of the second body member 14 and are positioned to align with the opening or openings of the first body member 12 opposite the nasal prongs 20A and 20B. The effective sizes of the openings to the inner volume of the second body member 14 and thus to the nasal prongs 20A and 20B of the first body member 12 provided by the FiO2 adjustment apertures 25A and 25B are individually adjustable. For instance, the sizes of the openings provided by the FiO2 adjustment apertures 25A and 25B in the depicted embodiment are adjustable from being closed to fully open and any condition therebetween by a sliding, rotation, or other actuation of adjustable aperture covers 26A and 26B.

The second body member 14 has a gas reception aperture 22 at a first end thereof. The gas reception aperture 22 fluidically engages the ambient pressure tubing 122 of the oxygen dispensing and conservation system 100, such as by being threadedly or otherwise engaged therewith or with a connector interposed between the ambient pressure tubing 122 and the nasal cannula 10. For instance, the second body member 14 in the present embodiment has a threaded tubular portion for being inserted into a distal end of the ambient pressure tubing 122 or a connector associated therewith. The second body member 14 further includes a one-way expiratory valve 24 disposed at a second end thereof in fluidic communication with the inner volume of the second body member 14. For instance, the second body member 14 in the present embodiment has a tubular portion at a second end thereof with the one-way expiratory valve 24 retained at the distal end of that tubular portion.

Under this configuration, the first and second body members 12 and 14 can be assembled as is illustrated, for example, in FIGS. 6-8 to have the nasal prongs 20A and 20B aligned with the corresponding opening or openings in the second body member 14. By virtue of the flexible nature of the first body member 12, a sealing engagement is effectively created between the nasal prong platform 30 and the nasal prongs 20A and 20B that project therefrom and the underlying surface of the second body member 14, which can be planar. Additionally, the first and second FiO2 adjustment apertures 25A and 25B and the first and second adjustable aperture covers 26A and 26B are exposed and accessible to the user for selective adjustment of entrained air. The one-way expiratory valve 24 is disposed in fluidic communication with the inner volume of the second body member 14 and, derivatively, the nasal prongs 20A and 20B.

In use with the oxygen dispensing and conservation system 100, therefore, the ambient pressure tubing 122 can be connected to the gas reception aperture 22 of the nasal cannula 10, whether directly or through a connector. The nasal cannula 10 can be retained relative to the head of the patient, such as by use of the straps 16 and the buckles 18, with the nasal prongs 20A and 20B received into the patient’s nares. Oxygen or any other gas retained by the donor reservoir 104 can then be readily inhaled through the nasal prongs 20A and 20B. The sizes of the openings provided by the FiO2 adjustment apertures 25A and 25B can be readily adjusted by operation of the first and second adjustable aperture covers 26A and 26B to provide a desired entrainment of atmospheric air with the oxygen or other gas as it is inhaled. Furthermore, during expiration, expired breath can be discharged directly through the cannula 10 through the one-way expiratory valve 24. Discharge of exhaled breath into the ambient pressure tubing 122 and consequent re-breathing of the same are thus prevented.

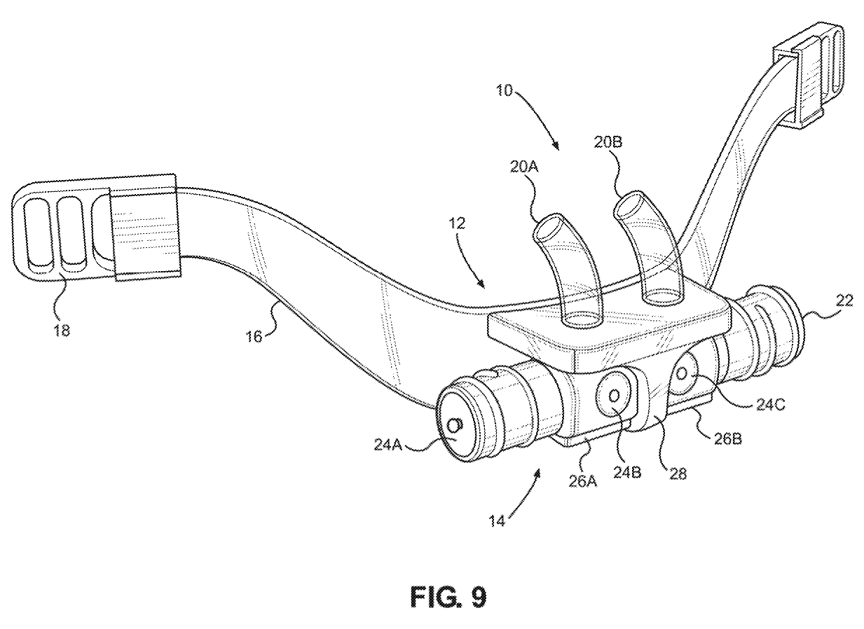

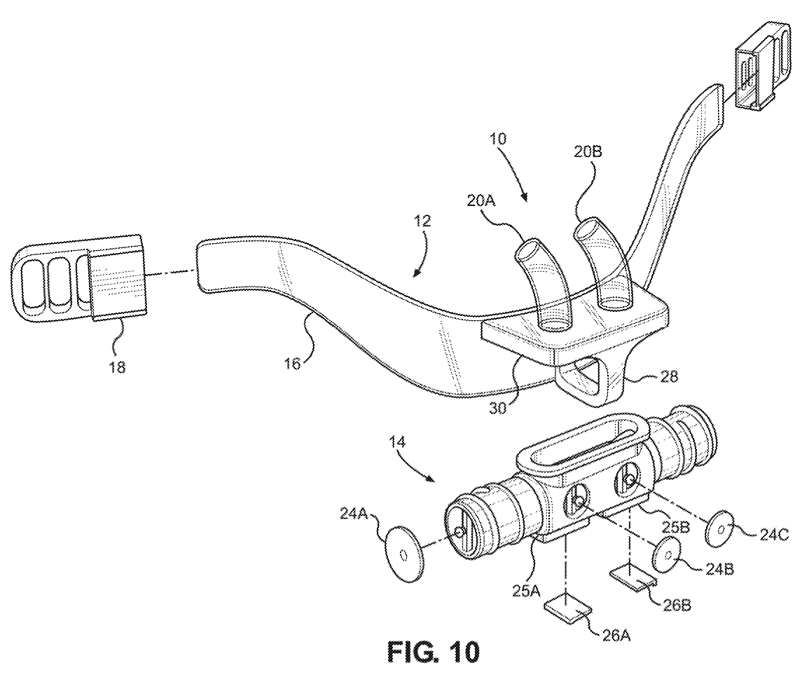

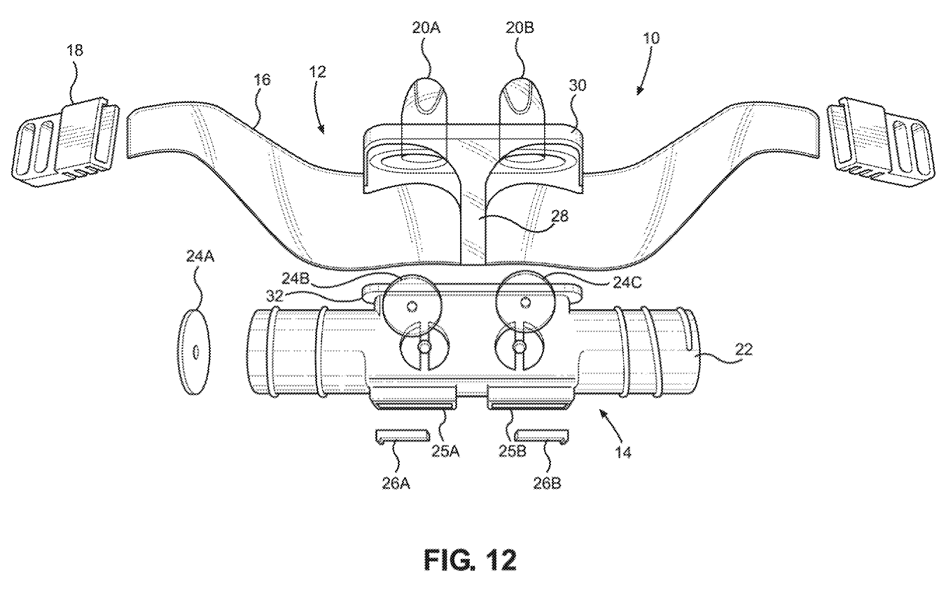

An alternative embodiment of the nasal cannula 10 for use with the oxygen dispensing and conservation system 100 is shown in FIGS. 9-12. There, the nasal cannula 10 again has a first body member 12 that is assembled with a second body member 14. The first body member 12 is again formed from a relatively flexible material while the second body member 14 is formed from a relatively rigid material. The central portion again has a nasal prong platform 30 from which first and second nasal prongs 20A and 20B project. The first body member 12 again has a central portion that defines a reception cavity. In the present embodiment, the reception cavity is formed with a resilient strap reception structure 28 in combination with the nasal prong platform 30. The second body member 14, which again is generally tubular, can be received through and retained within the reception cavity by the resilient strap reception structure 28 and the nasal prong platform 30 with a central portion of the second body member 14 passing through the reception cavity.

First and second straps 16 again extend laterally in opposite directions from the central portion of the first body member 12, and buckles 18 are retained at the distal ends of the straps 16. The nasal cannula 10 can then be retained relative to the head of a wearer by the straps 16 and buckles 18, again possibly in combination with an additional fastening mechanism or mechanisms, such as a head or neck strap, medical tape, or some other method or combination thereof. The first and second body members 12 and 14 are again interchangeably connected for removal and replacement of either or both components and to suit particular patient needs and preferences.

The second body member 14 has an open inner volume with an opening bounded by a platform 32 disposed to align with the nasal prong platform 30 and the nasal prongs 20A and 20B of the first body member 12 when the body members 12 and 14 are mutually engaged. The second body member 14 has first and second FiO2 adjustment apertures 25A and 25B that are in fluidic communication with the inner volume of the second body member 14. Here, the effective sizes of the openings to the inner volume of the second body member 14 provided by the FiO2 adjustment apertures 25A and 25B are individually adjustable by operation of sliding aperture covers 26A and 26B that enable the sizes of the openings provided by the FiO2 adjustment apertures 25A and 25B to be adjusted from being closed to fully open and to any condition therebetween by a sliding or other adjustment of the aperture covers 26A and 26B.

A gas reception aperture 22 is disposed at a first end of the second body member 14 for fluidically engaging the ambient pressure tubing 122 of the oxygen dispensing and conservation system 100 either directly, such as by being threadedly received into a distal end of the ambient pressure tubing 122 or by being engaged with a connector disposed therebetween. A first one-way expiratory valve 24A is disposed at a second end of the second body member 14 in fluidic communication with the inner volume of the second body member 14. The second body member 14 in this embodiment further includes second and third one-way expiratory valves 24B and 24C centrally disposed therealong and in fluidic communication with the inner volume of the second body member 14 and with the nasal prongs 20A and 20B when the first and second body members 12 and 14 are assembled. The second and third one-way expiratory valves 24B and 24C are disposed in general alignment with the nasal prongs 20A and 20B when the first and second body members 12 and 14 are assembled.

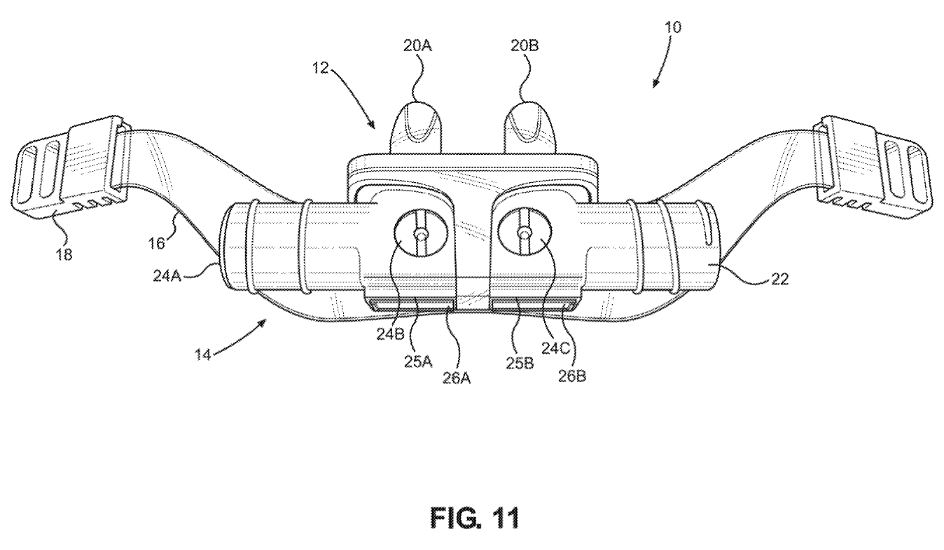

The first and second body members 12 and 14 can thus be assembled as shown, for instance, in FIGS. 9 and 11 to have the nasal prongs 20A and 20B aligned with the corresponding opening or openings in the second body member 14 and with the platform 32 of the second body member 14 aligned with the nasal prong platform 30 of the first body member 12. By virtue of the flexible nature of the first body member 12, a sealing engagement is effectively created between the nasal prong platform 30 and the nasal prongs 20A and 20B that project therefrom and the platform 32 of the second body member 14. Additionally, the first and second FiO2 adjustment apertures 25A and 25B are exposed and accessible to the user for selective adjustment by a sliding or other adjustment of the aperture covers 26A and 26B. The one-way expiratory valves 24A, 24B, and 24C are disposed in fluidic communication with the inner volume of the second body member 14 and, therethrough, with the nasal prongs 20A and 20B.

With the nasal cannula 10 so assembled, ambient pressure tubing 122 of the oxygen dispensing and conservation system 100 can be connected to the gas reception aperture 22 of the nasal cannula 10. The nasal cannula 10 can be caused to be retained relative to the head of the patient with the nasal prongs 20A and 20B received into the patient’s nares. The patient can then freely inhale oxygen or any other gas retained by the donor reservoir 104 through the nasal prongs 20A and 20B. Moreover, the sizes of the openings provided by the FiO2 adjustment apertures 25A and 25B can be readily adjusted by a simple sliding of the aperture covers 26A and 26B to control the mixture of atmospheric air with the inhaled oxygen. Breath expired by the patient can be discharged through the one-way expiration valves 24A, 24B, and 24C thereby preventing discharge into the ambient pressure tubing 122.

As discussed previously, while high-flow nasal cannula systems rely on the gaps around the nasal prongs of the cannula to vent excess flow even during inspiration, such an architecture is generally incompatible with permitting a patient to inspire naturally from an ambient pressure reservoir. In the ambient pressure system taught by the applicant, the gaps relied upon in prior art high flow cannulas provide an undesirable low-resistance path for the entrainment of ambient air. That entrainment of ambient air leads to unintended FiO2 dilution. Therefore, it is thus desirable in an ambient pressure system as taught by the inventors where oxygen is drawn on demand from an ambient pressure reservoir through a nasal cannula to prevent the unintended entrainment of ambient air through gaps around the nasal prongs of the cannula. Concomitantly, it is also desirable to permit the recipient to be able to exhale through the nares, including through the cannula itself.

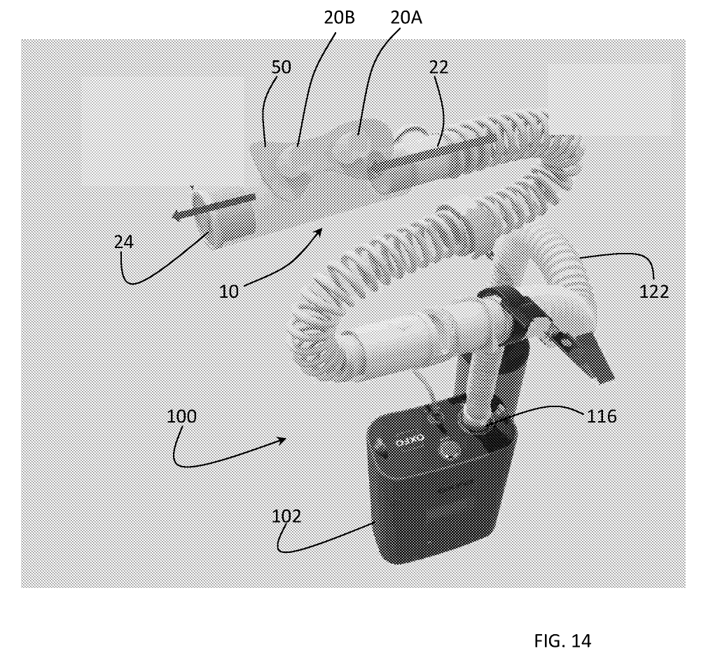

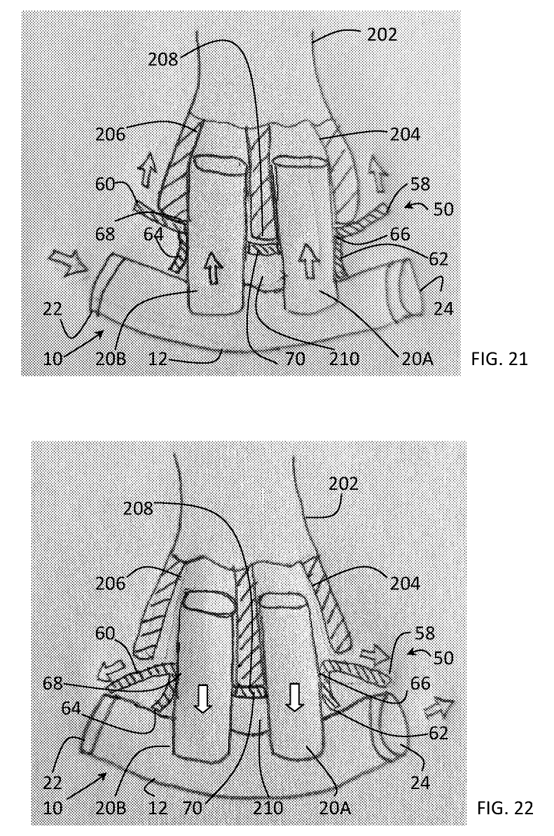

Recognizing these needs, the present inventors devised of the spring lamina valve indicated generally at 50 in FIGS. 14-22. As illustrated, the spring lamina valve 50 is engaged with the nasal cannula 10 to prevent the undesirable entrainment of ambient air from around the nasal prongs 20A and 20B during inhalation through the nares 204 and 206 of the nose 202 as depicted in FIG. 21 but to permit ready exhalation through the nares 204 and 206 through and around the nasal prongs 20A and 20B as in FIG. 22. As referenced herein, the term “lamina” should be interpreted as comprising a thin leaf of material. Within the scope of this, “lamina” is not limited to a single layer of material nor is it limited to a consistent thickness, to a varying thickness, or in any other way.

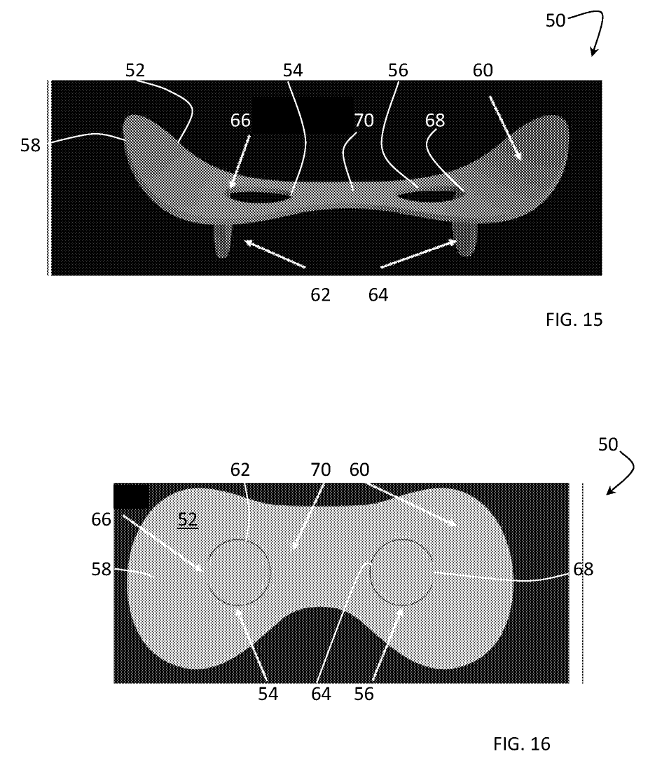

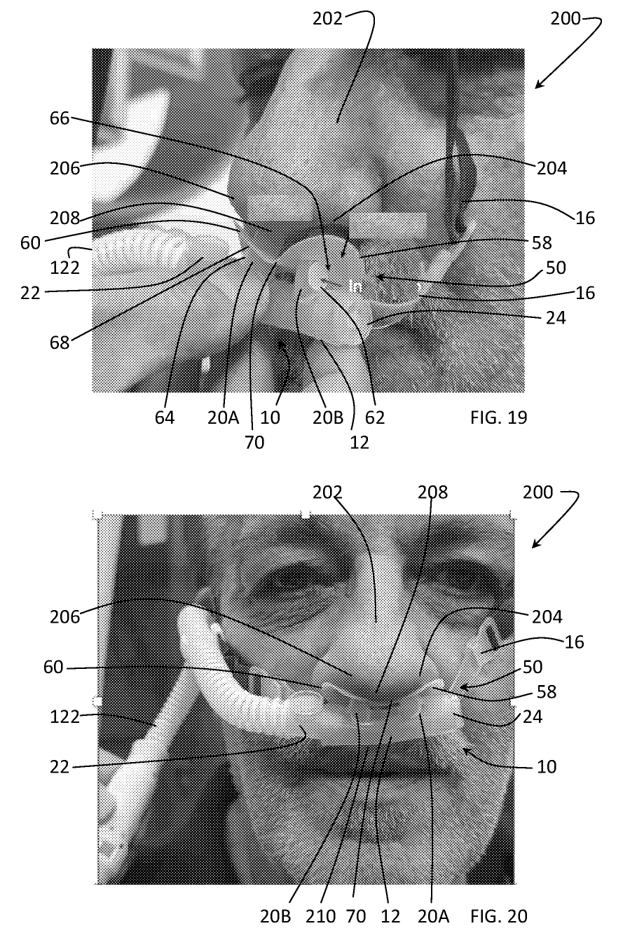

The spring lamina valve 50 is shown apart from the nasal cannula 10 in FIGS. 15 and 16. The spring lamina valve 50 has a main body 52 that is formed from a resiliently flexible, biocompatible material. In the present embodiment, the spring lamina valve 50 comprises a single body 52 for preventing undesirable entrainment of ambient air through both nares 204 and 206, but it would be possible for the lamina valve 50 to be formed with first and second bodies for engaging the left and right nares 204 and 206 as shown and described further hereinbelow. Here, as FIGS. 19 and 20 show, the spring lamina valve 50 has a left outer flap 58 for aligning with the left nare 204 of a patient 200 and a right outer flap 60 for aligning with the right nare 206 of the patient 200. The left outer flap 58 is joined with the right outer flap 60 by a bridge portion 70.

The spring lamina valve 50 is ergonomically sized and shaped to engage the nares 204 and 206, the columella 208 of the nose 200 therebetween, and the philtrum 210 below the nose 200 comfortably. As such, each outer flap 58 and 60 has a bulbous shape that generally corresponds to the shape of the respective nare 204 or 206. Each of the outer flaps 58 and 60 is proportionally greater in width, depth, and throughout its entire periphery than the nare 204 or 206 relative to which it is to be applied thereby to encompass the opening of each nare 204 and 206 entirely. Spring lamina valves 50 can be of varying sizes to accommodate differently sized and shaped noses 202. Alternatively, spring lamina valves 50 can be sized to be sufficiently large to accommodate all or substantially all likely nose sizes.

In this embodiment where the spring lamina valve 50 is unitary, a bridge portion 70 connects the outer flaps 58 and 60. The spring lamina valve 50 has an inner edge that is configured to approximate the shape of the upper lip, including the philtrum 210. As such, the inner edge of the spring lamina valve 50 is indented to pursue a generally sinusoidal shaped indentation centered around the bridge portion 70 to accommodate the shape of the upper lip and philtrum 210.

A left nasal prong aperture 54 is disposed in a central portion of the left outer flap 58, and a right nasal prong aperture 56 is disposed in a central portion of the right outer flap 60. The left and right nasal prong apertures 54 and 56 are sized and shaped in correspondence to the sizes and shapes of the nasal prongs 20A and 20B of the nasal cannula 10 relative to which the spring lamina valve 50 is to be applied such that the nasal prongs 20A and 20B are closely received in a fluid-tight or substantially fluid-tight manner. With that, ambient air is prevented or substantially prevented from flowing between the nasal prongs 20A and 20B and the outer flaps 58 and 60 of the spring lamina valve 50.

Under this construction, the spring lamina valve 50 can be applied to the nasal cannula 10 as, for instance, in FIGS. 14 and 17-22 with the left and right nasal prongs 20A and 20B inserted through the left and right nasal prong apertures 54 and 56, respectively. As illustrated in FIGS. 19 and 20, the now-joined nasal cannula 10 and spring lamina valve 50 can be applied to a patient 200 by causing the left and right nasal prongs 20A and 20B to be received at least partially into the left and right nares 204 and 206, respectively, until the spring lamina valve 50 is in contact with the lower surfaces of the nose 202 of the patient 200, such as with the left and right outer flaps 58 and 60 in contact or in proximity with the left and right nares 204 and 206 and the bridge portion 70 in contact or in proximity with the columella 208, the descending narrow strip of skin and cartilage that separates the nostrils or nares 204 and 206.

So retained and disposed, the resilience of the spring lamina valve 50 enables its performance as illustrated in FIGS. 21 and 22. The spring lamina valve 50 is configured to limit or prevent the uncontrolled dilution of oxygen drawn through the nasal cannula 10 with ambient air drawn through the nares 204 and 206 from around the left and right nasal prongs 20A and 20B during inhalation through the nares 204 and 206 as in FIG. 21 while the spring lamina valve 50 permits exhalation through the nares 204 and 206 as in FIG. 22.

During inhalation through the nares 204 and 206 as in FIG. 21, the left and right outer flaps 58 and 60 of the resilient spring lamina valve 50 will tend to be drawn by the negative inhalation pressure of the flowing gas into a general sealing engagement with the lower surfaces of the nares 204 and 206. With that, free entry of ambient air into the nares 204 and 206 through the spacing between the nasal prongs 20A and 20B and the nasal mucosa within the nares 204 and 206 is limited or entirely prevented. The fraction of inspired oxygen (Fi02) provided to the patient 200 can thus be reliably controlled, whether through adjustment apertures of the nasal cannula 10 as previously shown and described or through the composition of the gas supplied through the ambient pressure tubing 122.

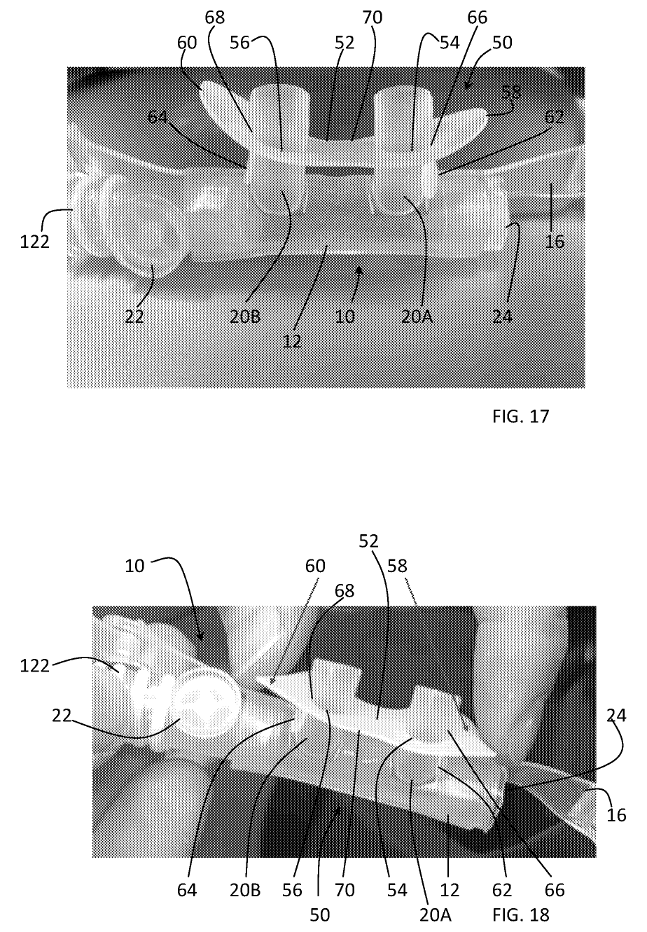

It will be noted from, for example, FIGS. 17 and 18 that the nasal prongs 20A and 20B in the depicted embodiment are larger than typical prior art nasal prongs. This is intentionally so. The nasal prongs 20A and 20B are intended to be as large as possible without providing an uncomfortable compression or other impact on the mucosae of the nares 204 and 206. With such a sizing, unintended oxygen entrainment is further prevented.

During exhalation through the nares 204 and 206 as in FIG. 22, at least some of the exhaled gas will flow along a reverse path through the nasal prongs 20A and 20B, through the expiratory valve or valves 24, and out of the nasal cannula 10. Concomitantly, the pressure of the exhaled gas will tend to bow the left and right outer flaps 58 and 60 out of contact with the skin at the ends of the nares 204 and 206 as FIG. 22 illustrates. This deflection of the outer flaps 58 and 60 enables a flow of exhaled gas through the spacing between the nasal mucosa and the left and right nasal prongs 20A and 20B. In certain cases, the patient 200 can further facilitate such a flow by flaring the nostrils 204 and 206 as FIG. 22 further depicts. When the exhalation by the patient ends, the left and right outer flaps 58 and 60 will tend to spring back into their undeflected shapes in or near close contact with the skin at the distal ends of the nares 204 and 206, and unintended entrainment of ambient air through the nares 204 and 206 will again be prevented.

With such a spring lamina valve 50 and a nasal cannula 10 incorporating such a spring lamina valve 50 designed, the present inventors appreciated that the ideal would be to have the main body 52 of the valve 50 to have a resting contour prior to deflection by the breathing flow of the patient that generally matches the contour of the lower surface of the nose 200 of the patient 200. As such, the main body 52 of the valve 50 would ideally have a resting contour when installed relative to the nasal cannula 10 that matches the contour presented by the lower surfaces of the nares 204 and 206 and the intervening columella 208, which pursue a generally saddle-shaped contour with a lower central area at the bridge portion 70 and smoothly, arcuately upturned outer distal ends of the left and right outer flaps 58 and 60. With that, the spring lamina valve 50 and the left and right outer flaps 58 and 60 will be at rest in contact or at least in near proximity to the lower surfaces of the nares 204 and 206 of the nose 200.

This could be accomplished by molding or otherwise forming the main body 52 of the spring lamina valve 50 to have a contoured shape with arcuately upturned outer distal ends of the left and right outer flaps 58 and 60. Such a manifestation of the spring lamina valve 50 is within the scope of the invention and the claims except as expressly excluded by the claims.

Recognizing the desirability of forming a spring lamina valve 50 with a main body 52 having a saddle-shaped contour with smoothly, arcuately upturned outer end portions, the inventors made a surprising discovery during crafting prototype spring lamina valves 50 from flat, resilient material seeking to carry forth the invention. The inventors had been using a hole punching tool as is commonly used to punch holes in leather goods to form the apertures 54 and 56 for receiving the nasal prongs 20A and 20B. In one instance, the hole punching tool left the chad, which would otherwise have been entirely separated from the main body 52, partially connected to the main body 52 over a small angular range along a laterally outer portion of the aperture. The chad was thus left as a flap that remained connected to the main body 52 over an angular range. Based on the resilience of the material forming the main body 52 and the flap, the flap was resiliently connected to the main body 52.

When the spring lamina valve 50 with the flap still resiliently attached to the main body 52 was applied to a nasal cannula 10 with the flap projecting toward the main body 12 of the cannula 10, a surprising thing happened: the adjacent outer flap naturally bowed to have a smoothly, arcuately upturned outer portion. When such a chad or flap was left partially attached to the main body 52 by a connection along a laterally outer portion of each aperture 54 and 56 and the spring lamina valve 50 so formed was applied to the nasal cannula 10 with the chads or flaps projecting toward the main body 12 of the cannula 10, it was found that the main body 52 of the spring lamina valve 50 naturally pursued the very saddle shape with smoothly upturned outer end portions that is desirable for closely matching and engaging the lower skin surfaces of the nares 204 and 206 and the columella 208.

It was further found that the chads or flaps so positioned prevented the unintended sliding displacement of the spring lamina valve 50 along the nasal prongs 20A and 20B, acting much like brakes and physical impediments to unintended displacement. To resist such unintended displacement, it would additionally or alternatively be possible within the scope of the invention for the nasal prongs 20A and 20B to have matching retaining formations therealong configured to maintain the spring lamina valve 50 at a desired location along the nasal prongs 20A and 20B. For instance, the nasal prongs 20A and 20B could have annular ridges or indentations or series of such annular ridges or indentations to permit a selective seating placement of the spring lamina valve 50 relative to the nasal prongs 20A and 20B and thus relative to the nasal cannula 10 in general.

The surprising, beneficial effect of the hanging flaps in bowing the outer flaps 58 and 60 is exploited in the embodiment of the spring lamina valve 50 depicted in FIGS. 14-22. There, the hanging flaps, which may be referred to as inner flaps, are indicated at 62 and 64. The inner flaps 62 and 64 in this embodiment are formed of the same resilient material as the main body 12 and the outer flaps 58 and 60 and are connected thereto by resilient connecting portions spanning a given angular range of the circular shapes of the apertures 54 and 56. Those resilient connecting portions, which may be referred to as isthmuses, are indicated at 66 and 68. The isthmuses 66 and 68 are disposed at the laterally outer edges of the circular shapes of the apertures 54 and 56 acting as hinges between the inner flaps 62 and 64 and the outer flaps 58 and 60.

With the inner flaps 62 and 64 and the isthmuses 66 and 68 so positioned, a deflection of the inner flaps 62 and 64 in a first direction will tend to bow the laterally outer portions of the outer flaps 58 and 60 in the opposite direction based on the resiliency of the material. Moreover, when the nasal prongs 20A and 20B are inserted through the apertures 54 and 56 in a close relationship with the isthmuses 66 and 68 spanning an arcuate angular range, a mechanical bowing effect is further realized by the constriction of the material of the main body 52 and the isthmuses 66 and 68 about the nasal prongs 20A and 20B.

It will be further observed, particularly with reference to FIGS. 20-22, that the nasal cannula 10 and the nasal prongs 20A and 20B thereof in this embodiment are further crafted to provide a comfortable ergonomic fit and to promote the sealing and unsealing characteristics of the valve 50 as described herein. More particularly, it can be seen that the main body 12 of the nasal cannula 10 pursues a V-shape, albeit with a large angle between the legs of the V. With this, the main body 12 of the nasal cannula 10 corresponds to the V-shape of the lower surfaces of many human noses. Moreover, the nasal prongs 20A and 20B themselves are angled toward one another from the proximal to the distal ends thereof, again in correspondence to the structure of a typical human nose where at least the outer mucosae of the nares 204 and 206, which are typically angled toward one another from the most distal end of the nares 204 and 206.

With this, applying the spring lamina valve 50 to the nasal cannula 10 with the nasal prongs 20A and 20B inserted through the apertures 54 and 56 with the inner flaps 62 and 64 projecting toward the main body 12 of the nasal cannula 10 will cause the main body 52 of the spring lamina valve 50 to be biased to the saddle shape with smoothly upturned laterally outer portions and a relatively lower bridge portion 70 that largely matches the contour of the lower surfaces of the nares 204 and 206 and the columella 208. A close yet comfortable fit is achieved. The outer flaps 58 and 60 will be resiliently drawn inwardly on inspiration through the nose 202 to limit or prevent the uncontrolled dilution of oxygen drawn through the nasal cannula 10 with ambient air drawn through the nares 204 and 206 from around the left and right nasal prongs 20A and 20B as in FIG. 21, and the outer flaps 58 and 60 are capable of flexing out of immediate contact with the nares 204 and 206 during exhalation as in FIG. 22 to permit exhalation through the nares 204 and 206 around and through the nasal prongs 20A and 20B. The spring lamina valve 50 thus provides selectable expiratory resistance and/or PEEP. The restoring force of the resilient material of the spring lamina valve 50 opposes expiratory flow to a selectable degree while still allowing safe venting and preventing undesired pressure accumulation. Through the material memory provided by the resilience of the spring lamina valve 50, the valve 50 will tend to spring back to its undeflected contour on the cessation of a deflecting force applied thereto, whether that be during inhalation or exhalation.

The spring lamina valve 50 can be formed from any material or combination of biocompatible materials that are ideally chosen to provide the resiliency and resultant sealing effects described herein. Non-limiting materials include biocompatible, flexible polymers, such as but not limited to silicone, polyethylene, and thermoplastic elastomers or polyurethane, polypropylene, or polyethylene films. Plural materials can be used in combination, including as composites, in laminated form, and otherwise. Spring lamina valves 50 can be manufactured in varied thicknesses and geometries within the scope of the invention, including to provide different sealing forces, different FiO2 modulation levels, and varied expiratory resistance contributions. Antimicrobial or hydrophilic coatings may be applied for patient comfort and safety. By selection of resilient materials with differing resistances to deflection, by differing thicknesses of the same resilient material, or by some combination thereof or by some other variation in construction, spring lamina valves 50 can be crafted to provide differing resistances to deflection on expiration thereby potentially providing differing positive end-expiratory pressure (PEEP) resistances.

The spring lamina valve 50 can have a consistent thickness or varying thickness to achieve desired performance characteristics. In certain embodiments, the spring lamina valve 50 can be formed from a panel of material with a thickness of approximately 0.05 mm to approximately 2.00 mm. Outer portions of the outer flaps 58 and 60 could be of a decreased thickness to add flexibility. The spring lamina valve 50 can decrease in thickness progressively or at specific locations. The thicknesses, angular ranges, and other characteristics of the isthmuses 66 and 68 can likewise be varied to produce varied flexions and varied resistances to flexion of the inner flaps 62 and 64 relative to the outer flaps 58 and 60.

Spring lamina valves 50 can be marked, color coded, or otherwise provided with indications of their differing flexion characteristics, such as with color coding, numbering, or some other indication of expiratory resistance/PEEP levels. By way of example and not limitation, a spring lamina valve 50 providing 1 cm H₂O resistance might be yellow while a spring lamina valve 50 providing 5 cm H₂O might be great, and so on. Spring lamina valves 50 can be manufactured in varied shapes and sizes to accommodate different patient anatomies and, potentially, to accommodate differing nasal cannulas 10, particularly the nasal prongs 20A and 20B thereof. Spring lamina valves 50 can be combined in kits with spring lamina valves 50 of the kit having different flexion characteristics.

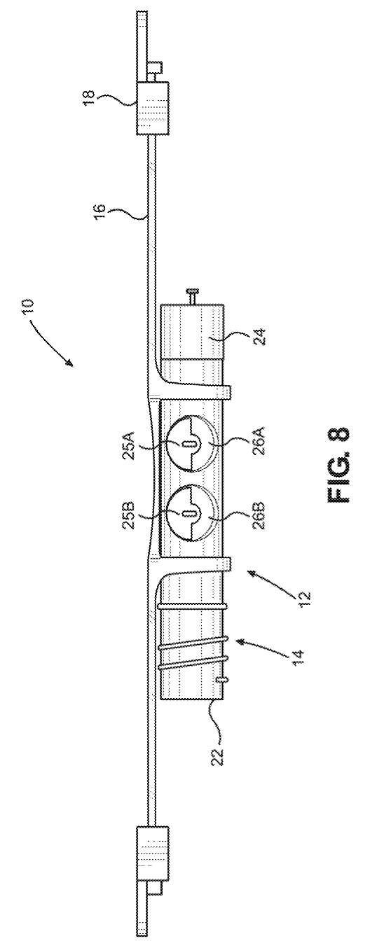

When coupled to a nasal cannula 10, the spring lamina valve 50 thus operates as an entrainment-control patient interface. When manufactured from a single planar lamina of silicone or other resilient, biocompatible material, the spring lamina valve 50 can, as FIG. 16 perhaps shows best, be considered to have a FIG. 8 shape with the left and right outer flaps 58 and 60 forming the upper and lower bulbous portions of the “8” and the bridge portion 70 connecting the two. The outer peripheral shape, the apertures 54 and 56 and the isthmuses 66 and 68 are precision cut to enable the valve 50 to self-form into a dynamic, anatomy-adaptive seal. The spring lamina valve 50 exhibits low-resistance, comfort-preserving sealing that suppresses unintended room-air entrainment during inhalation without requiring an intranasal plug seal and without reliance on positive pressure at the patient interface.

The outer flaps 58 and 60 are configured to conform to soft tissue around the nasal base and nare entrances, the inner flaps 62 and 64 act as dynamic valve leaflets, and the respective isthmuses 66 and 68 connecting the inner flaps 62 and 64 to the outer flaps 58 and 60 function as living hinges with elastic memory. Fabricated from biocompatible, elastomeric materials, such as but not limited to silicone or other polymers or elastomers, selected for comfort, fatigue resistance, and material memory, the lamina transitions from a flat condition as, for instance, in FIG. 16, into a preloaded spring geometry as, for instance, in FIG. 17 when mounted to the nasal prongs 20A and 20B of the nasal cannula 10. So mounted, the valve 50 exhibits a gentle sealing force that is automatically adaptive to the anatomy and motion of the patient 200.

The specific cut pattern of the lamina 50 defines independent compliant regions around each nare 204 and 206 and nasal prong 20A and 20B. Each isthmus 66 and 68 forms a controlled hinge axis that converts deflection of each respective inner flap 62 and 64 relative to each respective outer flap 58 and 60 into a repeatable restoring force. A preload mechanism is thus created when the inner flaps 62 and 64 are pivoted inwardly and the apertures 54 and 56 are pushed over and along the nasal prongs 20A and 20B to be interposed between the left and right outer flaps 58 and 60 and the main body 12 of the nasal cannula 10. The flaps 58, 60, 62, and 64 and the isthmuses 66 and 68 thus bend and store elastic energy. This spring loading transforms the low-cost planar part from which the spring lamina 50 is formed and the nasal cannula 10 relative to which it is coupled into active, spring-like entrainment-control elements without a need for discrete mechanical components, pins, or separate springs yielding both novel function and exceedingly effective industrial manufacturability.

Practices of the invention may thus be alternatively characterized as a method for preventing the entrainment of ambient air around the nasal prongs 20A and 20B of a nasal cannula 10 in an ambient pressure system 100 while permitting the passage of exhaled gases around those nasal prongs 20A and 20B during exhalation. In such a method, the ambient pressure system 100, the spring lamina valve 50, and the nasal cannula 10 may be as shown and described such that the method may be considered to be founded on providing each of the foregoing. The method may further include applying the spring lamina valve 50 to the nasal cannula 10 by causing the nasal prongs 20A and 20B to be received generally in parallel through the apertures 54 and 56 with the inner flaps 62 and 64 projecting from the main body 52 of the valve 50 toward the main body 12 of the cannula 10. Then, the nasal prongs 20A and 20B of the cannula 10 are inserted into the nares 204 and 206 of the patient 200, preferably until the outer flaps 58 and 60 are in immediate yet comfortable juxtaposition. With the spring lamina valve 50 so positioned, oxygen or other gas within the reservoir 104 is rendered available through the nasal cannula 10 with automatic sealing by the outer flaps 58 and 60 against unintended entrainment and comfortable exhalation around the nasal prongs 20A and 20B as needed.

As FIGS. 19 and 20 illustrate, when the spring lamina valve 50 is applied to a nasal cannula 10, the outer flaps 58 and 60 define broad, compliant, anatomy-conforming surfaces that rest against the nasal base and the nare entrance regions. The spring lamina valve 50 is sized and shaped to conform to a wide range of nose shapes in a manner that gently distributes contact pressure. The spring lamina valve 50 exhibits what may be referred to as self-compensation for spacing relative to the nares and for patient motion. With the resilient action of the spring lamina valve 50 generated by elastic deformation, the lamina valve 50 maintains gentle sealing contact even if, for instance, the cannula lamina valve 50 shifts slightly during speaking, jaw movement, head turning, or tubing tug or if the distance between the cannula body 12 and the nare entrances increases or decreases or if the nares 204 and 206 are not perfectly matching in shape. In certain embodiments, whether the outer flaps 58 and 60 are a unitary piece of material or as separate members, the configuration of the main body 52 of the spring lamina valve 50 permits each of the outer flaps 58 and 60 to be rotated or “clocked” about each nasal prong 20A and 20B independently to permit the flaps 58 and 60 to be settled at different angles. The lamina valve 50 can thus conform to asymmetric nares 204 and 206 and variable nare plane angles thereby enabling a fitting to noses 202 of substantially any shape and size without a need for multiple custom parts.

When the spring lamina valve 50 is in use with the present inventors’ ambient pressure system 100, the patient’s inspiratory effort creates negative pressure at the nares 204 and 206. Without the spring lamina valve 50, leakage paths would exist around the nasal prongs 20A and 20B thereby providing a route for room-air entrainment with resultant oxygen dilution and destabilized FiO2. With the spring-lamina valve 50 applied to the nasal cannula 50, however, negative inspiratory pressure pulls the outer flaps 58 and 60 toward the nare entrances and into closer apposition with the soft nasal tissue. Preloaded against the nasal prongs 20A and 20B, the inner flaps 62 and 64 transfer force to the outer flaps 58 and 60 through the isthmuses 66 and 68 thereby reinforcing the sealing effect precisely where leaks tend to occur around the bases of the nasal prongs 20A and 20B around the nare entrance perimeters. The result is dynamic sealing under suction such that the seal improves as the patient inhales harder, exactly when entrainment risk is highest.

During exhalation, the outer flaps 58 and 60 of the spring lamina valve 50 deflect thereby providing comfort-preserving relief through the exhaust paths around the nasal prongs 20A and 20B. In that regard, the nasal prongs 20A and 20B of the cannula 10 are thin walled and sized to fit within the nares 204 and 206 occupying a substantial portion of the inner circumference of the nares 204 and 206 while while avoiding uncomfortable wedging pressure against the nasal mucosa to permit prolonged wear. An annular clearance remains to preserve tissue perfusion and comfort. During exhalation, the lamina valve 50 relaxes and deflects thereby allowing limited flow through the annular clearance if and as needed while a main exhalation route is provided through the pathways provided by the nasal prongs 20A and 20B, the cannula body 12, and the expiratory valve 24. Low work of breathing and comfort are thus preserved while still entrainment during inhalation is suppressed.

Thus, the spring lamina valve 50 provides multiple advantages in use relative to an ambient-pressure system 10 as taught by the inventors, advantages that are uniquely desirable in an ambient-pressure environment where a volume of oxygen or perhaps another gas is retained for natural inhalation rather than being propelled in a high-flow environment. An effective seal is achieved while preserving nasal patency and minimizing mucosal pressure, which is critical for sick patients and long-duration oxygen therapy. Moreover, the seal improves automatically when inspiratory demand increases. Because the sealing action is driven by negative pressure and stored elastic energy, the interface self-tightens when the patient inhales harder, such as during peak inspiratory flows, thereby improving FiO2 stability exactly when most needed under high demand.

The spring lamina valve 50 and the nasal cannula 10 provide a universal fit across variable nare shapes and asymmetries. The precision cut geometry enables independent compliance and, perhaps, rotation per side, allowing one spring lamina valve 50 to adapt to non-coplanar nares and differing nare sizes thereby reducing the multiplicity of needed models and improving real-world usability. By avoiding sustained compressive wedging against mucosa, the spring lamina valve 50 supports blood flow, humidification, reduced ischemia, reduced pain, and better tolerance over hours of use. Where formed from a flat sheet, multiple spring lamina valves 50 can be manufactured at scale from a single sheet of silicone or other appropriate material, such as through die-cutting, laser cutting, stamping, or molding, with precisely repeatable dimensions. Resilient spring functionality is achieved simply by geometry and material memory, without a need for mechanical springs or multi-piece valve assemblies.

The spring lamina valve 50 and the nasal cannula 10 retaining the same are uniquely fitted for use in an ambient pressure system 10 while structures fitted for constant, high-flow systems would be poor fits in an ambient pressure system 10. For instance, CPAP fixed vents provide always-open exhaust ports intended for positive pressure, but these would become entrainment ports in an ambient-pressure reservoir system 10 thereby defeating FiO2 stability. Retained external to the nares 204 and 206 with the nasal prongs 20A and 20B received therethrough, the spring lamina valve 50 also distinguishes markedly from Clark-type intranasal plug-seals, which are designed to prevent air entry enirely by sealing circumferentially inside the nare in compressive mucosal contact.

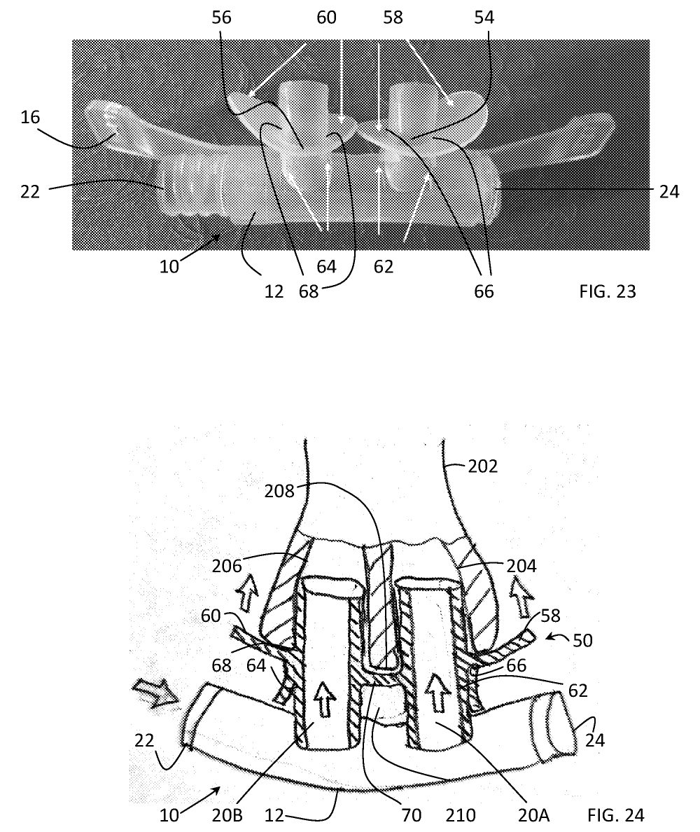

Other embodiments of the spring lamina valve 50 are possible and within the scope of the invention except as expressly excluded by the claims. For instance, as FIG. 23 illustrates, embodiments of the spring lamina valve 50 are contemplated wherein separate bodies form the left and right outer flaps 58 and 60. In this example, the separate left and right outer flaps 58 and 60 are again crafted from a panel of biocompatible, resilient material. Each of the left and right outer flaps 58 and 60 has a peripheral shape corresponding to the shape of the distal end of a nare 204 and 206 but marginally larger to cause the flaps 58 and 60 to overlie the skin at the bottom of each nare 204 and 206. The flaps 58 and 60 can again vary in size to accommodate different noses 202, or the flaps 58 and 60 can be crafted to fit a range of expected nose sizes. The left outer flap 58 has an aperture 54 centrally disposed therethrough for receiving the left nasal prong 20A of the cannula 10, and the right outer flap 60 has an aperture 56 therethrough for receiving the right nasal prong 20B of the cannula 10.

The outer flaps 58 and 60 of the spring lamina valve 50 again exploit the inventors’ discovery with regard to resilient inner flaps 62 and 64 being left connected to the outer flaps 58 and 60 by resilient isthmuses 66 and 68 at the peripheries of the apertures 54 and 56 to cause the outer flaps 58 and 60 to pursue the illustrated bowl-shaped configurations. Each of the inner flaps 62 and 64 is resiliently connected to the respective outer flap 58 or 60 by an isthmus 66 or 68. In the present embodiment, however, first and second inner flaps 62 and 64 are resiliently connected by isthmuses 66 and 68 to the respective outer flap 58 or 60 in opposition to each side of each aperture 54 and 56. Stated alternatively, two inner flaps 62 are resiliently connected in opposition to the left outer flap 58 over an angular range bounding the left aperture 54, and two inner flaps 64 are resiliently connected in opposition to the right outer flap 60 over an angular range bounding the right aperture 56. The angular range can vary within the scope of the invention. In one currently preferred embodiment, the range is approximately 60 degrees with that range being substantially centered at the laterally outermost tangent of the circular shapes of the prong apertures 54 and 56. Under this construction, when the spring lamina valve 50 is applied to a nasal cannula 10 by causing the left and right nasal prongs 20A and 20B to be received through the respective apertures 54 and 56 of the outer flaps 58 and 60 with the inner flaps 62 and 64 projected toward the main body 12 of the nasal cannula 10, the outer flaps 58 and 60 will be induced to the bowl-shaped configurations illustrated in FIG. 23 by the resilient effect of the pairs of inner flaps 62 and 64 and the connecting isthmuses 66 and 68 and, potentially, by the mechanical levering force of the isthmuses 66 and 68 over their angular ranges.

Again, the spring lamina valve 50 can be formed from any material or combination of biocompatible materials that provide the resiliency and resultant sealing effects described herein. For instance, biocompatible, flexible polymers can be employed, including silicone, polyethylene, and thermoplastic elastomers or polyurethane, polypropylene, or polyethylene films. As previously described, plural materials can be used in combination, including as composites, in laminated form, and otherwise. Spring lamina valves 50 with separate outer flaps 58 and 60 can be manufactured in varied thicknesses and geometries to provide different sealing forces, different FiO2 modulation levels, and varied expiratory resistance contributions. The materials of the spring lamina valve 50 can have a consistent thickness or varying thickness with certain embodiments again formed from a panel of material with a thickness of approximately 0.05 mm to approximately 2.00 mm. Outer portions of the outer flaps 58 and 60 could be of a decreased thickness to add flexibility whether through a progressive decrease in thickness progressively or by a stepping down of thickness at predetermined locations. As previously described, spring lamina valves 50 can be marked, color coded, or otherwise provided with indications of their differing flexion characteristics or some other indication of expiratory resistance/PEEP levels.

In each of the above-described embodiments, the ability to attach a separate spring lamina valve 50 to a nasal cannula 10 provides certain advantages, including the ability to apply spring lamina valves 50 of differing resistances to a given nasal cannula 10 to adapt to, for instance, different patient characteristics and needs. However, to be complete, it will be recognized that the spring lamina valve 50 could be fixedly retained relative to the nasal cannula 10. For instance, the spring lamina valve 50 could be integrally formed with the nasal cannula 10, or the spring lamina valve 50 could be fixed thereto, such as by adhesive, by plastic welding, or by any other method or combination thereof.

Such an embodiment is illustrated in FIG. 24. There, the spring lamina valve 50 is integrated with the nasal prongs 20A and 20B and thus with the nasal cannula 10 in general. The spring lamina valve 50 again has left and right outer flaps 58 and 60, and left and right inner flaps 64 and 66 are again resiliently hinged to the outer flaps 58 and 60 by isthmuses 66 and 68. It would also be possible to form the spring lamina valve 50 without such inner flaps 64 and 66 and instead to rely on a direct resilient connection between the outer flaps 58 and 60 and the nasal prongs 20A and 20B for similar effect in inducing the outer flaps 58 and 60 into the desired upturned configuration. A bridge portion 70 of the spring lamina valve 50 again spans between the outer flaps 58 and 60. Additionally or alternatively, the outer flaps 58 and 60 can be initially formed with a desired contour where the outer lateral portions and perhaps the anterior portions thereof are upturned to facilitate a close sealing contact with the lower ends of the nares 204 and 206 and the columella 208.

So positioned, the spring lamina valve 50 again prevents the undesirable entrainment of ambient air from around the nasal prongs 20A and 20B during inhalation as FIG. 24 illustrates, and the spring lamina valve 50 again permits ready exhalation through the nares 204 and 206 through and around the nasal prongs 20A and 20B as in FIG. 22 as the outer flaps 58 and 60 resiliently bow away from the nares 204 and 206 in response to the expiratory pressure imparted by the patient 200.

Within the scope of the invention, other embodiments of the spring lamina valve 50 are possible with resilient, variable-pressure biasing mechanisms for preventing or otherwise affecting the entrainment of ambient air through a nasal cannula 10. Each of the embodiments discussed hereinbelow exerts controlled, variable pressure over the openings of the nares 204 and 206 during patient inhalation to minimize or prevent unintended room-air entrainment when the patient 200 is drawing oxygen from an ambient-pressure reservoir 104. Each embodiment of the spring lamina valve 50 operates by dynamically increasing sealing force during inspiratory negative pressure and relaxing during exhalation or low-flow states. As below, the operative mechanism of the spring lamina valve 50 may, for instance, be passive, elastic, magnetic, pneumatic, or mechanically biased. In each instance, the material composition, geometry, thickness, surface characteristics, structural arrangement, and other elements may be adjusted to achieve the desired respiratory performance goals, including the reduction of ambient air entrainment, the maintenance of prescribed FiO2, the reduction of inspiratory dilution, the preservation of nasal comfort and mucosal integrity, low work of breathing, and fatigue resistance and repeatable elastic return. In each instance, the main body 52 of the spring lamina valve 50 can have a preferred thickness between approximately 0.1 mm and 3.0 mm and an even more preferable thickness between approximately 0.5 mm to 2.0 mm. Preferable thickness can depend on multiple factors, including the selected material and required compliance characteristics. Moreover, thickness can vary within a single spring lamina valve 50 to provide graded flexibility or differential biasing effects. Suitable materials include, but are not limited to, medical-grade silicone, elastomers, thermoplastic polymers, polymer films, thin spring metals, composite laminates, foam structures, magnetically responsive elements, and combinations thereof.

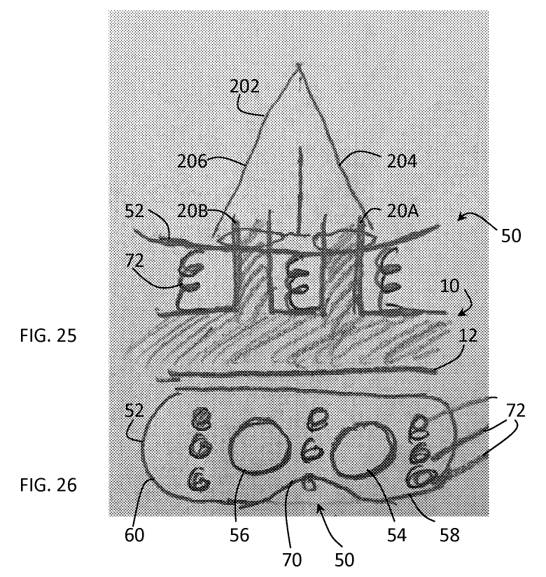

One alternative embodiment of the spring lamina valve 50 and a nasal cannula 10 engaging such a valve 50 is depicted in FIG. 25, and the spring lamina valve 50 is shown apart in FIG. 26. In the depicted embodiment, the main body 52 of the valve 50 is biased into resilient engagement with the nares 204 and 206 by coil springs 72 interposed at preferred locations between the main body 52 of the valve 50 and the main body 12 of the cannula 10. The main body 52 is again formed of flexible material. The coil springs 72 can comprise helical or micro-coil springs 72 can be attached to the main body 52 of the valve 50, to the main body 12 of the cannula 10, or to both. Here, coil springs 72 are disposed centrally to be positioned between the nasal prongs 20A and 20B and laterally outward of the nasal prongs 20A and 20B thereby to bias the outer flaps 58 and 60 and the bridge portion 70 of the valve 50 away from the main body 12 of the cannula and into engagement with the nares 204 and 206 of the patient 200.

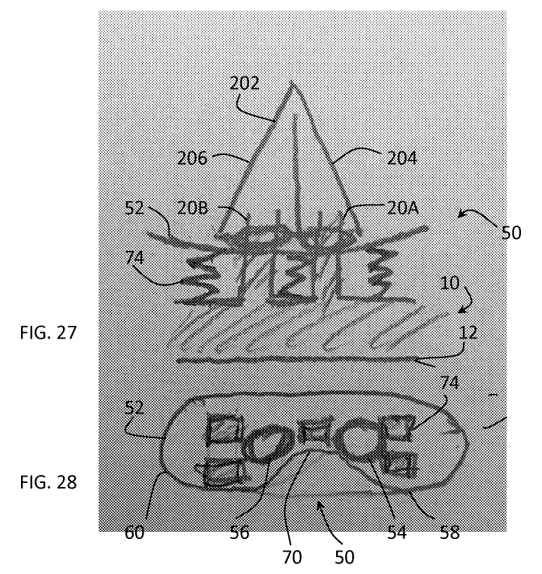

In the further embodiment of the spring lamina valve 50 and nasal cannula 10 shown in FIGS. 27 and 28, the main body 52 of the valve 50 is again flexible and biased into resilient engagement with the nares 204 and 206. Here, however, the biasing force is supplied by bellows springs 75 interposed at preferred locations between the main body 52 of the valve 50 and the main body 12 of the cannula 10. The bellows springs 74 again can be attached to the main body 52 of the valve 50, to the main body 12 of the cannula 10, or to both. A bellows springs 74 is disposed between the nasal prongs 20A and 20B. and pairs of bellows springs 74 are disposed laterally outward of the nasal prongs 20A and 20B thereby to bias the outer flaps 58 and 60 and the bridge portion 70 of the valve 50 away from the main body 12 of the cannula and into engagement with the nares 204 and 206 of the patient 200.

As FIGS. 29 and 30 show, the main body 52 of the spring lamina valve 50 can alternatively be biased into comfortable sealing contact with the nares 204 and 206 of the patient 200 by resiliently compressible open-cell or closed-cell compressible foam elements 76 interposed between the main body 52 of the valve 50 and the main body 12 of the cannula 10. Here, one foam element 76 is positioned to be disposed between the apertures 54 and 56 and thus between the nasal prongs 20A and 20B, and first and second larger foam elements 76 are positioned to be disposed laterally outward of the apertures 54 and 56 and the nasal prongs 20A and 20B. Thus, the central bridge portion 70 and the outer flaps 58 and 60 are resiliently biased toward the nares 204 and 206 of the patient 200.

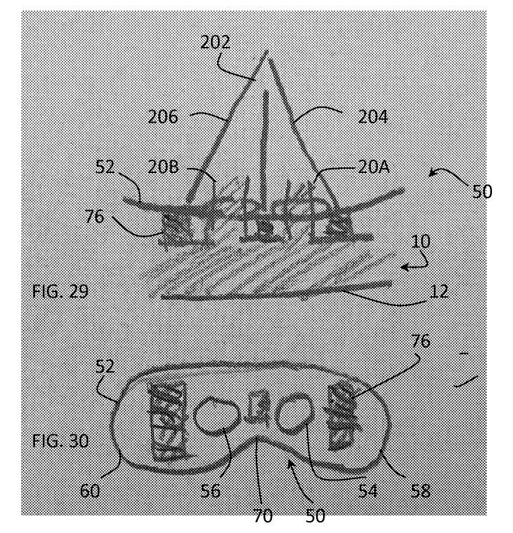

Numerous other mechanisms for biasing the main body 52 of the spring lamina valve 50, particularly the outer flaps 58 and 60 thereof, away from the main body 12 of the cannula 10 and into sealing engagement with the nares 204 and 206 are possible and within the scope of the invention. For instance, as in FIG. 31, one or more annular ribs 78 can be disposed along each of the nasal prongs 20A and 20B so that the main body 52 of the spring lamina valve 50 is maintained in contact with the nares 204 and 206 and prevented from sliding proximally along the nasal prongs 20A and 20B. In the depicted embodiment, there are three ribs 78 disposed in spaced relation along each of the nasal prongs 20A and 20B to enable a selective disposition of the main body 52 of the valve 50, but fewer or more ribs 78 are readily possible.

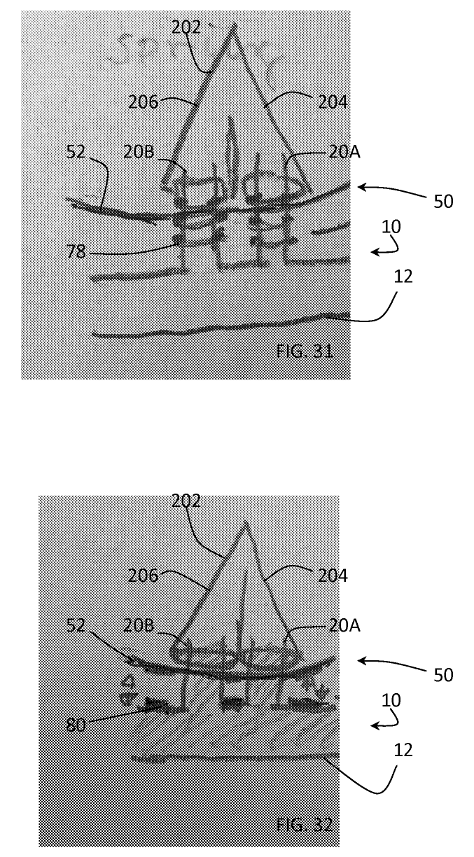

In another embodiment of the spring lamina valve 50 and nasal cannula 10 as in FIG. 32, a resilient biasing of the flexible main body 52 of the valve 50 is achieved by pairs of repelling magnets 80, each pair comprising one magnet 80 retained by the main body 12 of the nasal cannula 10 and a second magnet 80 retained by the main body 52 of the cannula 50. For instance, a first pair of repelling magnets 80 can be disposed between the nasal prongs 20A and 20B to resiliently bias the bridge portion 70 of the main body 52 of the nasal cannula 50 while one or more additional pairs of repelling magnets 80 are disposed outboard of the nasal prongs 20A and 20B to bias the outer flaps 58 and 60 toward the nares 204 and 206 of the patient 200.

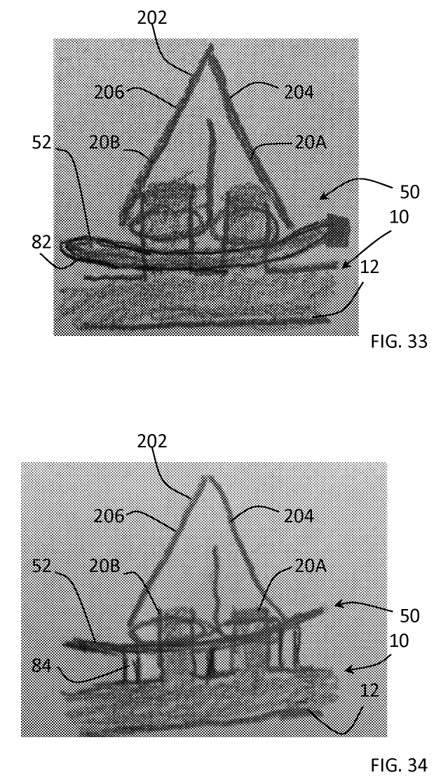

Looking to FIG. 33, yet another mechanism for biasing the spring lamina valve 50 into resilient, comfortable contact with the nares 204 and 206 of the patient 200 comprises a biasing balloon 82 interposed between the main body 52 of the spring lamina valve 50 and the main body 12 of the nasal cannula 10. In certain embodiments, the biasing balloon 82 comprises a then inflatable, elastomeric, or self-expanding membrane. The biasing balloon 82 could be a separate member from the main body 52 of the lamina valve 50, or the main body 52 of the lamina valve 50 could be partially or completely formed by a wall portion of the biasing balloon 82.

Yet another biasing configuration is illustrated in FIG. 34. There, a plurality of resiliently flexible columns 84, such as micro-columns, are interposed between the main body 52 of the spring lamina valve 50 and the main body 12 of the nasal cannula 10. Each of the columns 84 has a first end configured to press against the main body 52 of the spring lamina valve 50, a second end configured to press against the main body 12 of the nasal cannula 10, and a body portion traversing therebetween. The columns 84 can be of differing lengths to provide a contouring of the main body 52 of the spring lamina valve 50.

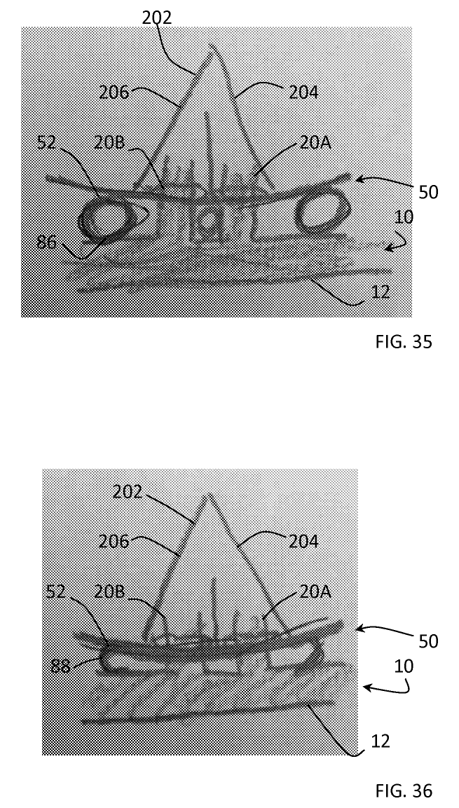

Further still, the spring lamina valve 50 can be biased into engagement with the nares 204 and 206 by tubular members, such as those indicated at 86 in FIG. 35 or those indicated at 88 in FIG. 36. In the manifestation of the spring lamina valve 50 of FIG. 35, three full tubular members 86 of resiliently compressible material are interposed between the main body 52 of the spring lamina valve 50 and the main body 12 of the nasal cannula 10 with a first tubular member 86 disposed between the nasal prongs 20A and 20B and with second and third tubular members 86 disposed laterally outward of the nasal prongs 20A and 20B. In the alternative manifestation of the spring lamina valve 50 of FIG. 36, semi-tubular members 88 of resiliently compressible material are interposed between the main body 52 of the spring lamina valve 50 and the main body 12 of the nasal cannula 10.

It is thus clear that the spring lamina valve 50 can pursue numerous different embodiments while blocking the entrainment of ambient air through the nares 204 and 206 and around the nasal prongs 20A and 20B. As shown and described herein, passive springs, arcuate lamina, flexible membranes, compressible rings, resilient collars, elastic diaphragms, segmented structures, or other spring-biased or resilient elements could be used to restrict or modulate air entry at the nares 204 and 206 while still accomplishing the same entrainment-control function. Spring-based and spring-biased elements are possible as are elastic or resiliently deformable structures are within the scope of the invention as are annular, radial, circumferential, axial, or segmented geometries. Structures disclosed herein dynamically respond to inspiratory pressure differentials. In perhaps the broadest sense, the present invention encompasses passive entrainment-limiting structures that surround the nasal prongs 20A and 20B with a first configuration upon inhalation sealing the nares 204 and 206 and preventing ambient air entrainment and a second configuration upon exhalation permitting the passage of exhaled breath through the nares 204 and 206 around and through the nasal prongs 20A and 20B.

As used herein, references to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, for example, the term “or” should generally be understood to mean “and/or.” Recitation of ranges of values herein are not intended to be limiting, referring instead individually to any and all values falling within the range, unless otherwise indicated herein, and each separate value within such a range is incorporated into the specification as if it were individually recited herein. The use of examples or exemplary language, as in “such as” or the like, provided herein, is intended merely to better illuminate the embodiments and does not pose a limitation on the scope of the embodiments. No language in the specification should be construed as indicating any unclaimed element as essential to the practice of the embodiments. In the description, it is understood that terms such as “first,” “second,” “top,” “bottom,” “upper,” “lower,” and the like are words of convenience and are not to be construed as limiting terms.

Within the present disclosure, the term “approximately” may be considered to mean within 10% of the stated value. The term “substantially” as used herein may broadly be considered to be any deviation that does not meaningfully affect function, performance, or intended use. Only where the foregoing is considered to be insufficiently clear, “substantially” with respect to a material property, such as “substantially rigid,” should be considered to be any deviation that is within 20% of the exact material property specified, such as by exhibiting a deflection under load within 20% of what a perfectly rigid body would allow. With respect to coverage or inclusion, “substantially” should be considered to be at least 90% of total coverage or inclusion, and “substantially” with regard to visual and structural properties should be considered to be within a deviation of 10% from the specified property, such as within 10% of perpendicular, within 10% of being perfectly flat, or within 10% of ambient pressure.

With certain details and embodiments of the present inventions for a spring lamina valve 50, a nasal cannula 10, and an ambient-pressure oxygen dispensing and conservation system 100 with which they are operative disclosed, it will be appreciated by one skilled in the art that numerous changes and additions could be made thereto without deviating from the spirit or scope of the invention. This is particularly true when one bears in mind that the presently preferred embodiments merely exemplify the broader invention revealed herein. Accordingly, it will be clear that those with major features of the invention in mind could craft embodiments that incorporate those major features while not incorporating all of the features included in the preferred embodiments.

Therefore, the following claims shall define the scope of protection to be afforded to the invention. Those claims shall be deemed to include equivalent constructions insofar as they do not depart from the spirit and scope of the invention. It must be further noted that a plurality of the following claims may express, or be interpreted to express, certain elements as means for performing a specific function, at times without the recital of structure or material. As the law demands, any such claims shall be construed to cover not only the corresponding structure and material expressly described in this specification but also all legally-cognizable equivalents thereof.

1. A nasal cannula in combination with a spring lamina valve for use with a gas dispensing system with tubing for providing gas to an individual with left and right nares, the nasal cannula and spring lamina valve comprising:
a nasal cannula comprising a nasal cannula body with an inner volume, left and right nasal prongs that extend from the nasal cannula body, the left nasal prong configured for being received into the left nare of the individual and the right nasal prong configured for being received into the right nare of the individual, wherein the left and right nasal prongs are in fluidic communication with the inner volume of the nasal cannula body, a gas reception aperture in fluidic communication with the inner volume of the nasal cannula body for receiving gas from the tubing of the gas dispensing system, a one-way expiratory valve retained by the nasal cannula body wherein the one-way expiratory valve is in fluidic communication with the inner volume of the nasal cannula body;
a spring lamina valve comprising a left outer flap comprising a thin leaf of resiliently flexible, biocompatible material, a right outer flap comprising a thin leaf of resiliently flexible, biocompatible material, a left nasal prong aperture through the left outer flap configured for closely receiving the left nasal prong therethrough, and a right nasal prong aperture through the right outer flap configured for closely receiving the right nasal prong therethrough;
whereby gas provided by the tubing can be inhaled through the left and right nasal prongs, whereby, during expiration, expired breath can be discharged through the one-way expiratory valve, whereby, during inhalation, the left and right outer flaps of the spring lamina valve will tend to be deflected toward the left and right nares of the individual, and whereby, during exhalation, the left and right outer flaps of the spring lamina valve will tend to be deflected away from the left and right nares of the individual.

2. The nasal cannula and spring lamina valve of claim 1, wherein the left outer flap and the right outer flap comprise separate members.

3. The nasal cannula and spring lamina valve of claim 1, wherein the left outer flap and the right outer flap of the spring lamina valve are connected by a bridge portion to have a resting contour when the left and right nasal prongs are received through the left nasal prong aperture and the right nasal prong aperture respectively and a peripheral shape with a left distal end portion, a right distal end portion, and an inner edge for being disposed adjacent to the upper lip and philtrum of the individual.

4. The nasal cannula and spring lamina valve of claim 3, wherein the left outer flap, the right outer flap, and the bridge portion are unitarily formed and wherein the inner edge is indented to pursue a generally sinusoidal shaped indentation centered around the bridge portion configured to accommodate the upper lip and philtrum of the individual.

5. The nasal cannula and spring lamina valve of claim 3, wherein the resting contour of the spring lamina valve when the left and right nasal prongs are received through the left nasal prong aperture and the right nasal prong aperture respectively with an upturned left distal end portion, an upturned right distal end portion, and a lower central portion coincident with the bridge portion.

6. The nasal cannula and spring lamina valve of claim 3, wherein the spring lamina valve further comprises a left inner flap resiliently and hingedly coupled to the left outer flap along the left nasal prong aperture by a left flap isthmus and a right inner flap resiliently and hingedly coupled to the right outer flap along the right nasal prong aperture by a right flap isthmus whereby, when the left and right nasal prongs are respectively received through the left and right apertures, the left and right inner flaps will tend to bow the left and right outer flaps.

7. The nasal cannula and spring lamina valve of claim 6, wherein the left and right nasal prong apertures are round and wherein each of the left and right flap isthmuses spans an arcuate angular range.

8. The nasal cannula and spring lamina valve of claim 6, wherein the left inner flap is resiliently and hingedly coupled to the left outer flap by the left flap isthmus along a laterally outer portion of the left nasal prong aperture and wherein the right inner flap is resiliently and hingedly coupled to the right outer flap by the right flap isthmus along a laterally outer portion of the right nasal prong aperture.

9. The nasal cannula and spring lamina valve of claim 6, wherein the left outer flap, the left inner flap, the left flap isthmus, the right outer flap, the right inner flap, the right flap isthmus, and the bridge portion are formed from a single panel of resilient material.

10. The nasal cannula and spring lamina valve of claim 9, wherein the single panel of resilient material comprises a resilient polymeric material with a thickness of between approximately 0.05 mm and approximately 2.00 mm.

11. The nasal cannula and spring lamina valve of claim 9, wherein the spring lamina valve is coded based on expiratory resistance provided by the spring lamina valve.

12. The nasal cannula and spring lamina valve of claim 1, wherein the left outer flap and the right outer flap comprise separate members and further comprising a first left inner flap resiliently and hingedly coupled to the left outer flap along the left nasal prong aperture by a first left flap isthmus and a first right inner flap resiliently and hingedly coupled to the right outer flap along the right nasal prong aperture by a first right flap isthmus whereby, when the left and right nasal prongs are respectively received through the left and right apertures, the first left and right inner flaps will tend to bow the left and right outer flaps.

13. The nasal cannula and spring lamina valve of claim 12, further comprising a second left inner flap resiliently and hingedly coupled to the left outer flap along the left nasal prong aperture by a second left flap isthmus and a second right inner flap resiliently and hingedly coupled to the right outer flap along the right nasal prong aperture by a second right flap isthmus whereby, when the left and right nasal prongs are respectively received through the left and right apertures, the second left and right inner flaps will tend to bow the left and right outer flaps.

14. The nasal cannula and spring lamina valve of claim 13, wherein the first and second left flap isthmuses are disposed in general opposition and wherein the first and second right flap isthmuses are disposed in general opposition.

15. The nasal cannula and spring lamina valve of claim 1, further comprising an FiO2 adjustment aperture in the body of the nasal cannula in fluidic communication with the inner volume of the body of the nasal cannula wherein the FiO2 adjustment aperture is selectively adjustable in size whereby the size of the FiO2 adjustment aperture can be adjusted to provide a desired entrainment of atmospheric air with gas inhaled through the first and second nasal prongs.

16. The nasal cannula and spring lamina valve of claim 15, wherein the body of the nasal cannula is formed from a first body member formed from a resilient and flexible material assembled with a second body member formed from a rigid material, wherein the first body member has a central portion that defines a reception cavity, and wherein the second body member can be at least partially received into the reception cavity of the first body member.

17. The nasal cannula and spring lamina valve of claim 1, further comprising a compliant biasing structure configured for resiliently biasing the left and right outer flaps of the spring lamina valve away from the nasal cannula body.

18. The nasal cannula and spring lamina valve of claim 17, wherein the compliant biasing structure comprises a plurality of springs configured to be interposed between the left and right outer flaps of the spring lamina valve and the nasal cannula body.

19. The nasal cannula and spring lamina valve of claim 17, wherein the compliant biasing structure comprises at least one resiliently compressible foam member configured to be interposed between the left and right outer flaps of the spring lamina valve and the nasal cannula body.

20. The nasal cannula and spring lamina valve of claim 17, wherein the compliant biasing structure comprises at least one protuberating formation disposed on the left nasal prong of the nasal cannula and at least one protuberating formation disposed on the right nasal prong of the nasal cannula.

21. The nasal cannula and spring lamina valve of claim 17, wherein the compliant biasing structure comprises at least one pair of repelling magnets comprising a first magnet retained by the nasal cannula and a second magnet retained by the spring lamina valve.

22. The nasal cannula and spring lamina valve of claim 17, wherein the compliant biasing structure comprises a biasing balloon configured for being interposed between the spring lamina valve and the nasal cannula.

23. The nasal cannula and spring lamina valve of claim 22, wherein the left outer flap and the right outer flap of the spring lamina valve are formed by a wall of the biasing balloon.

24. The nasal cannula and spring lamina valve of claim 17, wherein the compliant biasing structure comprises at least one resiliently compressible tubular member configured to be interposed between the left and right outer flaps of the spring lamina valve and the nasal cannula body.

25. A spring lamina valve for application to a nasal cannula with a nasal cannula body and left and right nasal prongs that extend from the nasal cannula body in a gas dispensing system with tubing for providing gas to an individual with left and right nares, the spring lamina valve comprising:
a left outer flap comprising a thin leaf of resiliently flexible, biocompatible material, wherein the left outer flap has a left nasal prong aperture configured for closely receiving the left nasal prong of the nasal cannula therethrough;
a right outer flap comprising a thin leaf of resiliently flexible, biocompatible material, wherein the right outer flap has a right nasal prong aperture configured for closely receiving the right nasal prong of the nasal cannula therethrough;
whereby, when the spring lamina valve is applied to the nasal cannula with the left and right nasal prongs respectively disposed through the left and right nasal prong apertures of the spring lamina valve, the left and right outer flaps of the spring lamina valve will tend to be deflected toward the left and right nares of the individual during inhalation and the left and right outer flaps of the spring lamina valve will tend to be deflected away from the left and right nares of the individual during inhalation.

26. The spring lamina valve of claim 25, wherein the left outer flap and the right outer flap comprise separate members.

27. The spring lamina valve of claim 26, further comprising a first left inner flap resiliently and hingedly coupled to the left outer flap along the left nasal prong aperture by a first left flap isthmus and a first right inner flap resiliently and hingedly coupled to the right outer flap along the right nasal prong aperture by a first right flap isthmus whereby, when the left and right nasal prongs are respectively received through the left and right apertures, the first left and right inner flaps will tend to bow the left and right outer flaps.

28. The spring lamina valve of claim 27, further comprising a second left inner flap resiliently and hingedly coupled to the left outer flap along the left nasal prong aperture by a second left flap isthmus and a second right inner flap resiliently and hingedly coupled to the right outer flap along the right nasal prong aperture by a second right flap isthmus whereby, when the left and right nasal prongs are respectively received through the left and right apertures, the second left and right inner flaps will tend to bow the left and right outer flaps.

29. The spring lamina valve of claim 28, wherein the first and second left flap isthmuses are disposed in general opposition and wherein the first and second right flap isthmuses are disposed in general opposition.

30. The spring lamina valve of claim 25, wherein the left outer flap and the right outer flap of the spring lamina valve are connected by a bridge portion to have a resting contour when the left and right nasal prongs are received through the left nasal prong aperture and the right nasal prong aperture respectively and a peripheral shape with a left distal end portion, a right distal end portion, and an inner edge for being disposed adjacent to the upper lip and philtrum of the individual.

31. The spring lamina valve of claim 30, wherein the left outer flap, the right outer flap, and the bridge portion are unitarily formed and wherein the inner edge is indented to pursue a generally sinusoidal shaped indentation centered around the bridge portion configured to accommodate the upper lip and philtrum of the individual.

32. The spring lamina valve of claim 30, wherein the resting contour of the spring lamina valve when the left and right nasal prongs are received through the left nasal prong aperture and the right nasal prong aperture respectively with an upturned left distal end portion, an upturned right distal end portion, and a lower central portion coincident with the bridge portion.

33. The spring lamina valve of claim 30, further comprising a left inner flap resiliently and hingedly coupled to the left outer flap along the left nasal prong aperture by a left flap isthmus and a right inner flap resiliently and hingedly coupled to the right outer flap along the right nasal prong aperture by a right flap isthmus whereby, when the left and right nasal prongs are respectively received through the left and right apertures, the left and right inner flaps will tend to bow the left and right outer flaps.

34. The spring lamina valve of claim 33, wherein the left and right nasal prong apertures are round and wherein each of the left and right flap isthmuses spans an arcuate angular range.

35. The spring lamina valve of claim 33, wherein the left inner flap is resiliently and hingedly coupled to the left outer flap by the left flap isthmus along a laterally outer portion of the left nasal prong aperture and wherein the right inner flap is resiliently and hingedly coupled to the right outer flap by the right flap isthmus along a laterally outer portion of the right nasal prong aperture.

36. The spring lamina valve of claim 33, wherein the left outer flap, the left inner flap, the left flap isthmus, the right outer flap, the right inner flap, the right flap isthmus, and the bridge portion are formed from a single panel of resilient material.

37. The spring lamina valve of claim 36, wherein the single panel of resilient material comprises a resilient polymeric material with a thickness of between approximately 0.05 mm and approximately 2.00 mm.

38. The spring lamina valve of claim 37, wherein the spring lamina valve is coded based on expiratory resistance provided by the spring lamina valve.

39. The spring lamina valve of claim 25, further comprising a compliant biasing structure configured for resiliently biasing the left and right outer flaps of the spring lamina valve away from the nasal cannula body.

40. The spring lamina valve of claim 39, wherein the compliant biasing structure comprises a plurality of springs configured to be interposed between the left and right outer flaps of the spring lamina valve and the nasal cannula body.

41. The spring lamina valve of claim 39, wherein the compliant biasing structure comprises at least one resiliently compressible foam member configured to be interposed between the left and right outer flaps of the spring lamina valve and the nasal cannula body.

42. The spring lamina valve of claim 39, wherein the compliant biasing structure comprises at least one pair of repelling magnets comprising a first magnet configured to be retained by the nasal cannula and a second magnet retained by the spring lamina valve.

43. The spring lamina valve of claim 39, wherein the compliant biasing structure comprises a biasing balloon configured for being interposed between the spring lamina valve and the nasal cannula.

44. The spring lamina valve of claim 39, wherein the compliant biasing structure comprises at least one resiliently compressible tubular member configured to be interposed between the left and right outer flaps of the spring lamina valve and the nasal cannula body.
